(19) United States
(12) Patent Application Publication
(10) Pub. No.: US 2026/0355202 A1
(43) Pub. Date: Nov. 12, 2026

(54) Combination Therapies Comprising SHP2 Inhibitors and EGFR Tyrosine Kinase Inhibitors

(71) Applicant: HUYABIO International, LLC, San Diego, CA (US)

(72) Inventors: Farbod Shojaei, San Diego, CA (US); Jill M. Ricono, Encinitas, CA (US); Robert Goodenow, San Clemente, CA (US); Mireille Gillings, San Diego, CA (US)

(21) Appl. No.: 19/721,960

(22) Filed: Jun. 26, 2026

Related U.S. Application Data
(63) Continuation of application No. 17/736,869, filed on May 4, 2022.
(60) Provisional application No. 63/184,697, filed on May 5, 2021.
(60) Provisional application No. 63/320,991, filed on Mar. 17, 2022.

(51) Int. Cl. A61K 31/519; A61K 31/506; A61P 35/00

(52) U.S. Cl. CPC A61K 31/519; A61K 31/506; A61P 35/00

(57) ABSTRACT

Provided herein are combinations that include a SHP2 inhibitor and an EGFR TKI and methods of treating cancer.

### CROSS-REFERENCE TO RELATED APPLICATION(S)

This application is a continuation of U.S. patent application Ser. No. 17/736,869, filed May 4, 2022, which claims the benefit of U.S. Provisional Patent Application Ser. No. 63/184,697, filed May 5, 2021, and U.S. Provisional Application No. 63/320,991, filed Mar. 17, 2022, the contents of which are incorporated herein by reference in their entireties.

### TECHNICAL FIELD

The present disclosure relates to combinations comprising of a SHP2 and an EGFR tyrosine kinase inhibitor; and uses of such combinations in the treatment of cancers.

### BACKGROUND

Cancer is a significant cause of morbidity and mortality worldwide. While the standards of care for many different cancer types have greatly improved over the years, current standards of care still fail to meet the need for effective therapies to improve treatment of cancer. SH2-containing protein tyrosine phosphatase 2 (SHP2) belongs to the protein tyrosine phosphatase family, which is involved in regulating cell proliferation, survival, differentiation, migration and apoptosis. In recent years, SHP2, has been shown to play an important role in tumor inhibition, especially as the role of SHP2 in tumors has become increasingly clear. Therefore, activation of SHP2 has become a feasible anti-tumor strategy.

In the protein tyrosine phosphatase superfamily, SHP2 is the first true proto-oncogene to be confirmed, and it plays an important role in a variety of signaling pathways such as metabolism, differentiation, proliferation, migration and survival. SHP2 can regulate Ras-mitogen-activated protein kinase, Janus kinase-signal transducer and activator of transcription (JAK-STAT) or phosphoinositide 3-kinase-AKT and nuclear factor κB (NF-κB) and other signaling pathways. SHP2 is also the main regulator of the immune checkpoint signaling pathway of programmed cell death protein-1 (PD-1) and B and T lymphocyte attenuation factor (BTLA), which may be related to tumor immunosuppression. In addition, SHP2 mutations rarely occur in tumors.

The epidermal growth factor receptor (EGFR) is a transmembrane protein that is a receptor for members of the epidermal growth factor family (EGF family) of extracellular protein ligands. In many cancer types, mutations affecting EGFR expression or activity result in cancer. A number of drug treatments have been developed to target EGFR. One method involves using small molecules to inhibit the EGFR tyrosine kinase (TK), which is on the cytoplasmic side of the receptor. Without kinase activity, EGFR is unable to activate itself, which is a prerequisite for binding of downstream adaptor proteins. Ostensibly, by halting the signaling cascade in cells that rely on this pathway for growth, tumor proliferation and migration is diminished. However, many patients develop resistance. Two primary sources of resistance are the T790M Mutation and MET oncogene. Therefore, a significant number of tumors are either resistant or become refractory. Accordingly, there is a need for new therapies, including, for example, combination therapies for the treatment of cancers. Provided herein are combination and methods of treating cancer.

### SUMMARY

Provided herein, inter alia, are combinations comprising a SHP2 inhibitor and an EGFR tyrosine kinase (TK) inhibitor.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1A is a log-linear graph depicting the superior potency of HBI-2376 (compound of Formula IA) in a cell proliferation assay compared to TNO-155 and RMC-4550 in an in vitro HCC827-ER1 cell model. These results are summarized in Table 1 in Example 1.

FIG. 1B is a log-linear graph depicting the superior potency of HBI-2376 (compound of Formula IA) in a cell proliferation assay compared to TNO-155 and RMC-4550 in an in vitro NCI-H1975 cell model. These results are summarized in Table 1 in Example 1.

FIG. 2A and FIG. 2B are tumor volume graphs demonstrating that the compound of Formula (Ia) in combination with osimertinib is efficacious at reducing tumor volume.

FIG. 3 is an immunohistochemistry study of relative ERK and pERK expression.

FIG. 4 are representative tissue images of the conducted immunohistochemistry experiments.

FIG. 5 is an immunoblot representing expression of ERK and pERK in tumors after treatment with HBI-2376 (compound of Formula IA), osimertinib and RMC-4550.

FIG. 6 is an immunoblot representing expression of ERK and pERK after treatment with HBI-2376 (compound of Formula IA), osimertinib and RMC-4550.

FIG. 7 quantifies the relative expression of ERK and pERK in tumors after treatment with HBI-2376 (compound of Formula IA), osimertinib and RMC-4550.

FIG. 8 is an immunoblot representing expression of DUSP6 protein after treatment with HBI-2376 (compound of Formula IA), osimertinib and RMC-4550.

FIG. 9 quantifies the relative expression of DUSP6 in tumors after treatment with HBI-2376 (compound of Formula IA), osimertinib and RMC-4550.

FIG. 10A, FIG. 10B, FIG. 10C, and FIG. 10D are representative tissue images of the conducted immunohistochemistry experiments.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

In an aspect, provided herein is a combination comprising a SHP2 inhibitor compound of Formula (Ia), or a pharmaceutically acceptable salt or solvate thereof:

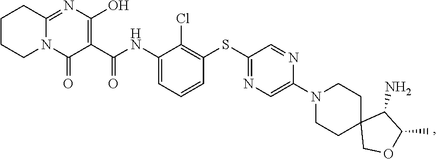 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

and an EGFR TK inhibitor.

In some embodiments, the combination comprises from about 5 mg to about 100 mg of the compound of Formula (Ia), or a pharmaceutically acceptable salt or solvate thereof. In some embodiments, the combination comprises about 5 mg, about 10 mg, about 20 mg, about 30 mg, about 40 mg, or about 50 mg of the compound of Formula (Ia), or a pharmaceutically acceptable salt or solvate thereof.

In some embodiments, the EGFR TK inhibitor is a small molecule compound, a nucleic acid, a peptide, a protein, an antibody, a peptibody, a diabody, a minibody, a single-chain variable fragment (ScFv), or a fragment or variant thereof. In some embodiments, the EGFR TK inhibitor is selected from erlotinib, afatinib, gefitinib, osimertinib, dacomitinib, icotinib, rociletinib, olmutinib, tarloxotinib, TAK-788, amivantamab (JNJ-6372), or AC0010. In some embodiments, the EGFR TK inhibitor is osimertinib.

In another aspect, provided herein is a pharmaceutical composition comprising a combination as described herein and a pharmaceutically acceptable excipient.

In another aspect provided herein are methods for preventing and/or treating non-receptor protein tyrosine phosphatase-mediated or dependent diseases or conditions. In some embodiments, provided herein is a method of treating cancer in a subject in need thereof, comprising administering to the patient a combination comprising a therapeutically effective amount of a compound of Formula (Ia), or a pharmaceutically acceptable salt or solvate thereof:

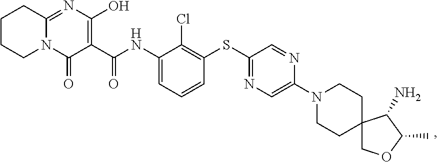 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

and a therapeutically acceptable amount of an EGFR TK inhibitor.

In some embodiments, the compound of Formula (Ia), or a pharmaceutically acceptable salt or solvate thereof, is administered to said patient in need from about 5 mg/kg to about 25 mg/kg. In some embodiments, the compound of Formula (Ia), or a pharmaceutically acceptable salt or solvate thereof is administered to said patient in need at about 5 mg/kg, about 10 mg/kg, about 15 mg/kg, about 20 mg/kg, or about 25 mg/kg.

In some embodiments, the EGFR TK inhibitor is a small molecule compound, a nucleic acid, a peptide, a protein, an antibody, a peptibody, a diabody, a minibody, a single-chain variable fragment (ScFv), or a variant thereof. In some embodiments, the EGFR TK inhibitor is selected from erlotinib, afatinib, gefitinib, osimertinib, dacomitinib, icotinib, rociletinib, olmutinib, tarloxotinib, TAK-788, amivantamab (JNJ-6372), or AC0010. In some embodiments, the EGFR TK inhibitor is Osimertinib.

In some embodiments, the method comprises administering the compound of Formula (Ia), or a pharmaceutically acceptable salt or solvate thereof; and the EGFR TK inhibitor simultaneously or sequentially. In some embodiments, the method comprises administering the compound of Formula (Ia), or a pharmaceutically acceptable salt or solvate thereof; and the EGFR TK inhibitor to the patient as a regimen. In some embodiments, the method comprises administering a compound of Formula (Ia), or a pharmaceutically acceptable salt or solvate thereof; and an EGFR TK inhibitor to said patient orally or as an intraperitoneal injection. In some embodiments, administration is by intravenous injection (I.V). In some embodiments, the method comprises administering a compound of Formula (Ia), or a pharmaceutically acceptable salt or solvate thereof; and an EGFR TK inhibitor to the patient daily. In some embodiments, the method comprises administering a compound of Formula (Ia), or a pharmaceutically acceptable salt or solvate thereof; and an EGFR TK inhibitor QD, BID, or TID.

In some embodiments, the patient is treatment naïve. In some embodiments, the method comprises administering a compound of Formula (Ia), or a pharmaceutically acceptable salt of solvate thereof; and an EGFR TK inhibitor to said patient as a first line therapy. In some embodiments, the method comprises administering a compound of Formula (Ia), or a pharmaceutically acceptable salt or solvate thereof; and an EGFR TK inhibitor to the patient as a second, third, fourth, fifth, or sixth line of treatment. In some embodiments, the method comprises administering a compound of Formula (Ia), or a pharmaceutically acceptable salt or solvate thereof; and an EGFR TK inhibitor to the patient following treatment with at least one anti-cancer therapy, wherein the anti-cancer therapy is chemotherapy, radiotherapy, surgery, targeted therapy, immunotherapy, or a combination thereof. In some embodiments, the method comprises administering a compound of Formula (Ia), or a pharmaceutically acceptable salt or solvate thereof; and an EGFR TK inhibitor to a patient who has failed at least one EGFR TK therapy. In some embodiments, the cancer is resistant to at least one anti-cancer agent.

In some embodiments, the cancer is squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

In some embodiments, the method comprises inhibiting metastasis of the cancer in the patient in need of treatment. In some embodiments, the method of treating cancer prolongs the time to disease progression of said cancer in the patient. In some embodiments, the method of treating cancer prolongs the survival of the patient. In some embodiments, the method of treating cancer increases progression-free survival of the patient. In some embodiments, the method of treating cancer reduces a tumor or tumor burden in the patient. In some embodiments, the method reduces or prevents metastasis of a primary tumor in the patient in need.

Other objects, features and advantages of the combinations and methods described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION

### Combinations

In one aspect, described herein are combinations (e.g., combination therapies, such as therapeutic methods and uses, kits, and compositions) for treating cancer. In some embodiments, the combinations described herein comprise a SHP2 inhibitor and an EGFR TK inhibitor. In some embodiments, a combination may comprise a first pharmaceutical composition and a second pharmaceutical composition. In some embodiments, the first pharmaceutical composition comprises a SHP2 inhibitor and the second pharmaceutical composition comprises an EGFR TK inhibitor. In some embodiments, the first pharmaceutical composition and the second pharmaceutical composition are co-packaged as a kit, which may further include instructions for co-administration of the first and second pharmaceutical compositions. In some embodiments, the first and second compositions may be packaged separately for combination in a clinical setting by administering them to a patient within a time frame during which the patient derives clinical benefit from the first pharmaceutical composition and the second pharmaceutical composition at the same time. In some embodiments, a combination may comprise a pharmaceutical composition comprising a SHP2 inhibitor and an EGFR TK inhibitor. In some embodiments, a combination comprises a unit dosage form of a pharmaceutical composition comprising a SHP2 inhibitor and an EGFR TK inhibitor. In some embodiments, a combination comprises a first pharmaceutical composition comprising a SHP2 inhibitor for use in the treatment of cancer in combination with a second pharmaceutical composition comprising an EGFR TK inhibitor. In some embodiments, a combination comprises a use of a SHP2 inhibitor for preparation of a first pharmaceutical composition for use in the treatment of cancer in combination with a second pharmaceutical composition comprising an EGFR TK inhibitor. In some embodiments, the SHP2 inhibitor is a pyrazine derivative, e.g., of Formula (I), Formula (Ia), Formula (II), Formula (III), Formula (IIIa), Formula (IV), or Formula (IVa), as described herein; and the EGFR TK inhibitor is any such inhibitor described herein, such as erlotinib, afatinib, gefitinib, osimertinib, dacomitinib, icotinib, rociletinib, olmatinib, tarloxotinib, TAK-788, amivantamab (JNJ-6372), or AC0010.

In some embodiments, described herein is a combination comprising:
(i) a therapeutically effective amount of a SHP2 inhibitor having the structure of Formula (I), or a pharmaceutically acceptable salt or solvate thereof:

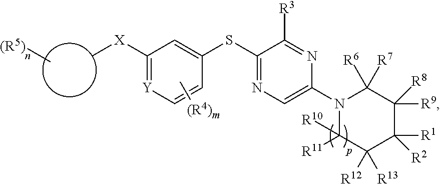

wherein,
   R¹ and R² are each the same or different, and they are each independently selected from H, D, halogen, —CN, —C(O)OH, —CHO, —OH, —NO₂, and the following substituted or unsubstituted groups:—NH₂. C₁-C₁₀ alkyl, C₁-C₁₀ alkylamino, C₁-C₁₀ alkoxy, C₃-C₁₂ cycloalkyl, C₃-C₁₂ cycloalkyloxy, 3-12 membered heterocyclic group, C₆-C₁₀ aryl group, 5-10 membered heteroaryl group; or R¹ and R² form a 3-8 membered saturated or unsaturated cycloalkyl or heterocyclic group, optionally, the 3-8 membered saturated or unsaturated cycloalkyl or heterocyclic group has one to three —OH, —NH₂, —CN, NO₂, halogen, C₁-C₁₀ alkyl, C₁-C₁₀ alkoxy, C₁-C₁₀ alkylamino, C₃-C₁₂ cycloalkyl, C₆-C₁₀ aryl or 5-10 membered heteroaryl;
   R³ is selected from H, D, or —NH₂;
   X is selected from a bond, —NH—, or —C(O)NH—;
   Y is selected from N or CR¹³, wherein R¹³ is selected from H, D, —OH, —CN, halogen, C₁-C₁₀ alkyl group, C₁-C₁₀ alkoxy, C₃-C₁₂ cycloalkane amino, C₁-C₁₀ alkylamino, C₃-C₁₂ cycloalkyl, 3-8 membered heterocyclic group, halogenated C₁-C₁₀ alkylamino, or C₆-C₁₀ aryl or 5-10 membered heteroaryl group, the heterocyclic group or heteroaryl group optionally contains one to four heteroatoms, and the heteroatoms are selected from S, O, N or NH;
   each R⁴ is the same or different, and is independently selected from H, D, halogen, —CN, —C(O)OH, —CHO, —OH, —NO₂, —C(O)NHR¹⁴ or —NHC(O)R¹⁵, substituted or unsubstituted with the following groups:—NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R¹⁴ and R¹⁵ are each independently selected from C₁-C₁₀ alkylamino, C₃-C₁₂ cycloalkyl, C₆-C₁₀ aryl or 5-10 membered heteroaryl; the substitution is selected from C₁-C₁₀ alkyl, halogen atom, —NH₂, —CN, —C(O)OH, —CHO, —OH, —NO₂, C₁-C₁₀ alkoxy, C₁-C₁₀ alkylamino, C₃-C₁₂ cycloalkyl, C₆-C₁₀ aryl, 5-10 membered heteroaryl or 3-12 membered heterocyclic group is substituted by one or more substituents, the above-mentioned substituents are optionally substituted with one to three substituents selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl;

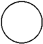

is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl, 3-12 membered heterocyclic group, C₆-C₁₄ bridged heterocyclic group or spiro heterocyclic group contains one to three heteroatoms or groups selected from N, NH, O, S, C(O), or S(O);
each R⁵ is the same or different, and is independently selected from H, D, halogen, —CN, —C(O)OH, —CHO, —OH, —NO₂, aminoacyl, substituted or unsubstituted following groups: C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substitution is selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl group or 3-12 membered heterocyclic group substituted by one or more substituents; or any two adjacent R⁵ form a 3-6 membered saturated or unsaturated ring, and optionally the 3-6 membered saturated or unsaturated ring is substituted with one to three-OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl;
R⁶, R⁷, R⁸, R⁹, R¹⁰, R¹¹, R¹², and R¹³ are each independently selected from H, D, halogen, —CN, —C(O)OH, —CHO, —OH, —NO₂, substituted or unsubstituted the group selected from —NH₂, C₁-C₁₀ alkyl, C₁-C₁₀ alkylamino, C₁-C₁₀ alkoxy, C₃-C₁₂ cycloalkyl, C₃-C₁₂ cycloalkyloxy group, 3-12 membered heterocyclic group, C₆-C₁₀ aryl, and 5-10 membered heteroaryl, the substitution is selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaryl or C₆-C₁₀ aryl;
m is 0, 1, 2 or 3;
n is 0, 1, 2 or 3; and
p is 0, 1, or 2; and
(ii) a therapeutically effective amount of an EGFR TK inhibitor.

In some embodiments, the compound of Formula (I) has the structure of Formula (II), or a pharmaceutically acceptable salt or solvate thereof:

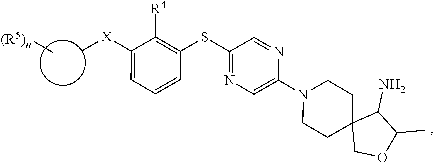

wherein,
   X is selected from chemical bond, —NH—, —CONH—;
   R⁴ is selected from H, D, halogen atom, —CN, —C(O)OH, —CHO, —OH, —NO₂, —C(O)NHR¹⁴ or —NHC(O)R¹⁵, substituted or unsubstituted with the group selected from —NH₂, C₁-C₁₀ alkyl, C₁-C₁₀ alkylamino, C₁-C₁₀ alkoxy, C₃-C₁₂ cycloalkyl, 3-12 membered heterocyclic group, C₆-C₁₀ aryl, or 5-10 membered heteroaryl; wherein R¹⁴ and R¹⁵ are each independently selected from C₁-C₁₀ alkylamino, C₃-C₁₂ cycloalkyl, C₆-C₁₀ aryl or 5-10 membered heteroaryl group; the substituent is selected from C₁-C₁₀ alkyl, halogen, —NH₂, —CN, —C(O)OH, —CHO, —OH, —NO₂, or C₁-C₁₀ alkoxy, substituted by one or more substituents of C₁-C₁₀ alkylamino, C₃-C₁₂ cycloalkyl, C₆-C₁₀ aryl, 5-10 membered heteroaryl, or 3-12 membered heterocyclic group, the substituents are optionally selected from C₁-C₁₀ alkyl, halogen, —NH₂, —CN, —C(O)OH, —CHO, —OH, —NO₂, C₁-C₁₀ alkoxy, C₁-C₁₀ alkylamino, or C₃-C₁₂ cycloalkyl;

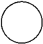

is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl group, 3-12 membered heterocyclic group, C₆-C₁₄ bridged heterocyclic group or spiro heterocyclic group contains one to three heteroatom or groups selected from N, NH, O, S, C(O), or S(O);
each R⁵ is the same or different, and is independently selected from H, D, halogen atom, —CN, —C(O)OH, —CHO, —OH, —NO₂, or aminoacyl, substituted or unsubstituted with the group selected from C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substituent selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl or 3-12 membered heterocyclic group substituted by one or more substituents; or any two adjacent R⁵ form a 3-6 membered saturated or unsaturated ring, and is optionally, the 3-6 membered saturated or unsaturated ring is comprises one to three-OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl; and
n is 0, 1, 2 or 3;
In some embodiments, R⁴ is selected from H, D, halogen, —CN, unsubstituted or halogen atom substituted C₁-C₁₀ alkyl.

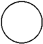

In some embodiments, is elected from phenyl, naphthyl, 5-10 membered heteroaryl or 3-12 membered heterocyclic group; wherein the 5-10 membered heteroaryl group and 3-12 membered heterocyclic group contain one to three heteroatoms or groups optionally selected from N, NH, O, S, or C(O).

In some embodiments, the 5-10 membered heteroaromatic ring is selected from thienyl; pyridyl; pyrimidinyl; pyrazinyl; pyridazinyl; pyrrolyl; pyrazolyl; thiazolyl; 1,2,3-triazolyl; 1,2,4-triazoly; imidazolyl; tetrazolyl; isothiazolyl; oxazolyl; isoxazolyl; thiadiazolyl; oxadiazolyl; benzothienyl; indolyl; benzimidazolyl; benzothiazolyl; benzofuranyl; quinolinyl; isoquinolinyl; quinazolinyl; indazolyl; indole[1,2-a]pyrazinyl; 4,7-diazaindole; pyrazolopyrimidinyl; imidazo-pyrimidinyl; oxazolopyrimidinyl; isoxazopyrimidiny; imidazopyrazinyl; pyrazolopyrazine; pyrrolopyrazinyl; or furan. In some embodiments, any one of pyrazinyl, thienopyrazinyl, pyridopyrimidinone, benzoxazolyl, and benzothiazolyl; the 3-12 membered heterocyclic group is selected from aziridinyl, oxetanyl, pyrrolidinyl, tetrahydrofuranyl, tetrahydrothienyl, piperidinyl, morpholinyl, piperazinyl, thiomorpholinyl, tetrahydropyranyl, 1,1-dioxythiomorpholinyl, butyrolactam, valerolactam, caprolactam, butyrolactone, valerolactone, caprolactone, succinimide or

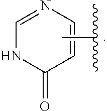

In some embodiments, the 3-12 membered heterocyclic group is selected from butyrolactamyl, pyrrolidinyl, succinimide, or

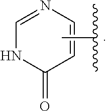

In some embodiments, each R⁵ is the same or different, and is independently selected from H, D, halogen, —CN, —C(O)OH, —CHO, —OH, —NO₂, or aminoacyl, substituted or unsubstituted with a C₁-C₁₀ alkyl, C₁-C₁₀ alkylamino, C₁-C₁₀ alkoxy, or —NH₂, and the substitution is selected from C₁-C₁₀ alkyl, halogen, —NH₂, —CN, —OH, —NO₂ are substituted by one or more substituents; or any two adjacent R⁵ form a 3-6 membered saturated or unsaturated ring, optionally, the 3-6-membered saturated or unsaturated ring is substituted with one to three-OH, —NH₂, —CN, halogen, C₁-C₁₀ alkyl, and C₁-C₁₀ alkoxy.

In some embodiments, the compound of Formula (I) has the structure of Formula (Ia), or a pharmaceutically acceptable salt or solvate thereof:

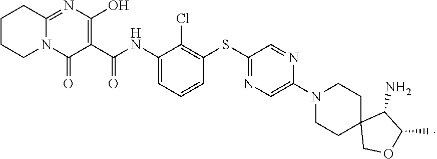 [SMILES: C[C@@H]1OCC2(CCN(c3cnc(Sc4cccc(NC(=O)c5c(O)nc6n(c5=O)CCCC6)c4Cl)cn3)CC2)[C@@H]1N]

In some embodiments, the compound of Formula (I) or (Ia) is N-(3-((5-((3S,4S)-4-amino-3-methyl-2-oxa-8-azaspiro[4.5]decan-8-yl) pyrazin-2-yl)thio)-2-chlorophenyl)-2-hydroxy-4-oxo-6,7,8,9-tetrahydro-4H-pyrido[1,2-a]pyrimidine-3-carboxamide, or a pharmaceutically acceptable salt or solvate thereof.

In some embodiments, the compound of Formula (I), (Ia), or (II), or a pharmaceutically acceptable salt thereof is a SHP2 inhibitor. The compound of Formulas (I), (Ia) and (II) are substantially described by International Patent Application No. PCT/CN2020/077391, filed Mar. 2, 2020, which is incorporated herein by reference in its entirety.

In some embodiments, described herein is a combination comprising:
(i) a therapeutically effective amount of a SHP2 inhibitor having the structure of Formula (III), or a pharmaceutically acceptable salt or solvate thereof:

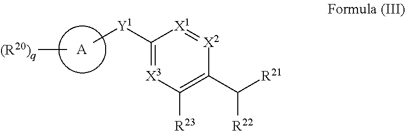

wherein,
   X¹ is N or CH;
   X² is N or CH;
   X³ is N or CH;
      wherein at least one of X¹, X², or X³ is N;
   Y¹ is S or direct bond;
   A is selected from the group consisting of 5- to 12-membered monocyclic or poly cyclic cycloalkyl, monocyclic or polycyclic heterocycloalkyl, monocyclic or polycyclic aryl, or polycyclic heteroaryl;
   R²⁰ is independently, at each occurrence, —H, -D, —C₁-C₆alkyl, —C₂-C₆alkenyl, —C₄-C₈acycloalkenyl, —C₂-C₆alkynyl, —C₃-C₈cycloalkyl, —OH, —OR²⁵, halogen, —NO₂, —CN, —NR²⁴R²⁵, —SR²⁴, —S(O)₂NR²⁴R²⁵, —S(O)₂R²⁴, —NR²⁴S(O)₂NR²⁴R²⁵, —NR²⁴S(O)₂R²⁵, —S(O)NR²⁴R²⁵, —S(O)R²⁴, —NR²⁴S(O)NR²⁴R²⁵, —NR²⁴S(O)R²⁵, —C(O)R²⁴, —CO₂R²⁴, —C(O)NR²⁴R²⁵, —NR²⁴CO) R²⁵, or 3- to 12-membered monocyclic or polycyclic heterocycle, wherein each alkyl, alkenyl, cycloalkenyl, alkynyl, cycloalkyl or heterocycle is optionally substituted with one or more-OH, halogen, —NO₂, oxo, —CN, —R²⁴, —OR²⁴, —NR²⁴R²⁵, —SR²⁴, —S(O)₂NR²⁴R²⁵, —S(O)₂R²⁴, —NR²⁴S(O)₂NR²⁴R²⁵, —NR²⁴S(O)₂R²⁵, —S(O)NR²⁴R²³, —S(O) R², —NR²⁴S(O)NR²⁴R²⁵, —NR²⁴S(O)R²⁵, heterocycle, aryl, or heteroaryl;
   R²¹ and R²² are independently selected from the group consisting of —H, -D, —OH, —C₁-C₆alkyl, a 3- to 12-membered monocyclic or polycyclic heterocycle, a 5- to 12-membered spiroheterocycle, C₃-C₈cycloalkyl, —(CH₂)_{q}—R²⁸, or —(CH₂)_{q}C(O)NR²⁴R²⁵, wherein each alkyl, heterocycle, or cycloalkyl is optionally substituted with one or more —C₁-C₆alkyl, —OH, —NH₂, —OR²⁸, —NHR²⁸, —(CH₂), OH, heterocyclyl, or spiroheterocyclyl;
   or R²² can combine with R²¹ to form a 3- to 12-membered monocyclic or polycyclic heterocycle, or a 5- to 12-membered spiroheterocycle, wherein each heterocycle or spiroheterocycle is optionally substituted with one or more —C₁-C₆alkyl, halogen, —OH, —OR, —NHR²⁸, optionally substituted heteroaryl, optionally substituted heterocyclyl, —(CH₂)_{q}NH₂, —(CH₂)_{q}OH, —COOR²⁸, —CONHR²⁸, —CONH(CH₂)_{q}COOR²⁸, —NHCOOR²⁸, —O—C(O)—NR²⁴R²⁵, —CF₃, —CHF₂, —CH₂F, or ═O; wherein the heteroaryl and heterocyclyl are optionally substituted with —CN;
   R²³ is —C₁-C₆alkyl, —C₁-C₆haloalkyl, —C₁-C₆hydroxyalkyl-CF₂OH, —CHFOH, —NH—NHR²⁴, —NH—OR²⁴, —O—NR²⁴R²⁵, —NHR²⁴, —OR²⁴, —NHC(O)R²⁴, —NHC(O)NHR²⁴, —NHS(O)₂R²⁴, —NHS(O)₂NHR²⁴, —S(O)₂OH, —C(O)OR²⁴, —NH(CH₂), OH, —C(O)NH(CH₂)ₙOH, —C(O)NH(CH₂)_{q}R²⁸, —C(O)R²⁸, —NH₂, —OH, —CN, —C(O)NR²⁴R²⁵, —S(O)₂NR²⁴R²⁵, C₃-C₈cycloalkyl, aryl, heterocyclyl containing 1-5 heteroatoms selected from the group consisting of N, S, P, and O, or heteroaryl containing 1-5 heteroatoms selected from the group consisting of N, S, P, and O, wherein each alkyl, cycloalkyl, or heterocyclyl is optionally substituted with one or more —OH, —NH₂, —OR, halogen, or oxo; wherein each aryl or heteroaryl is optionally substituted with one or more-OH, —NH₂, or halogen;
   R²⁴ and R²⁵ are each independently, at each occurrence, selected from the group consisting of —H, -D, —C₁-C₆alkyl, —C₂-C₆alkenyl, —C₃-C₈cycloalkenyl, —C₂-C₆alkynyl, —C₃-C₈cycloalkyl, a monocyclic or polycyclic 3- to 12-membered heterocycle, —OR²⁶, —SR²⁶, halogen, —NR²⁶R²⁷, —NO₂, and —CN;
   R²⁶ and R²⁷ are independently, at each occurrence, —H, -D, —C₁-C₆alkyl, —C₂-C₆alkenyl, —C₄-C₈cycloalkenyl, —C₂-C₆alkynyl, —C₃-C₈cycloalkyl, a monocyclic or polycyclic 3- to 12-membered heterocycle, wherein each alkyl, alkenyl, cycloalkenyl, alkynyl, cycloalkyl, or heterocycle is optionally substituted with one or more-OH, —SH, —NH₂, —NO₂, or —CN;
   R²⁸ is independently-H, -D, —C₁-C₆alkyl, —C₁-C₆cycloalkyl, —C₂-C₆alkenyl, —(CH₂)_{q}-aryl, heterocyclyl containing 1-5 heteroatoms selected from the group consisting of N, S, P, or O, or heteroaryl containing 1-5 heteroatoms selected from the group consisting of N, S, P, and O; wherein each alkyl, cycloalkyl, alkenyl, heterocycle, heteroaryl, or —(CH₂)_{q}-aryl is optionally substituted with one or more-OH, halogen, —NO₂, oxo, —CN, —R²⁴, —OR²⁴, —NR²⁴R²⁵, —SR²⁴, —S(O)₂NR²⁴R²⁵, —S(O)R²⁵, —NR²⁴S(O)₂NR²⁴R²⁵, —NR²⁴S(O)₂R²⁵, —S(O)NR²⁴R²⁵, —S(O)R²⁴, —NR²⁴S(O)NR²⁴R²⁵, —NR²⁴S(O)R²⁵, —C(O)NR²⁴R²⁵—, —NR²⁴R²⁵C(O)—, heterocycle, aryl, heteroaryl, (CH₂)_{q}OH, —C₁-C₆alkyl, CF₃, CHF₂, or CH₂F; and
   q is independently 0, 1, 2, 3, 4, 5, 6, 7, 8, 9, or 10; and
(ii) a therapeutically effective amount of an EGFR tyrosine kinase (TK) inhibitor.

In some embodiments, the compound of Formula (IIIa), or a pharmaceutically acceptable salt of solvate thereof:

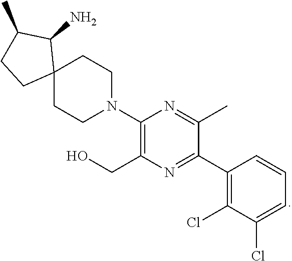 [SMILES: Cc1nc(N2CCC3(CC[C@@H](C)[C@H]3N)CC2)c(CO)nc1-c1cccc(Cl)c1Cl]

In some embodiments, the compound of Formula (IIIa) is RMC-4550 or a pharmaceutically acceptable salt or solvate thereof. In some embodiments, the SHP2 inhibitor is a compound described in PCT/US2018/055502 filed on Oct. 11, 2018, which is herein incorporated by reference in its entirety.

In some embodiments, described herein is a combination comprising:
(i) a therapeutically effective amount of a SHP2 inhibitor having the structure of Formula (IV), or a pharmaceutically acceptable salt or solvate thereof:

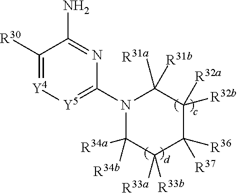

wherein
   c is selected from 0 and 1;
   d is selected from 0 and 1;
   Y⁴ is selected from CH and N;
   Y⁵ is selected from CR³⁵ and N;
   R³⁰ is-X⁵R^{30a}, wherein
      R^{30a} is selected from C₆₋₁₀aryl, C₃₋₈cycloalkyl, C₃₋₈Cycloalkenyl and a 5-9 member heteroaryl group containing from 1 to 4 heteroatoms or groups independently selected from N, C(O), O and S; wherein said aryl or heteroaryl of R^{30a} is substituted with 1 to 5 R³⁸ groups independently selected from halo, amino, hydroxy, N₃, C₁₋₄alkyl, dimethyl-amino, hydroxy-substituted-C₁₋₄alkyl, halo-substituted-C₁₋₄alkyl, amino-substituted-C₁₋₄alkyl, —C(O)OR⁴⁰ and —NHC(O)R⁴⁰; and
      X⁵ is selected from a bond, S(O)ₘ₁, O, C(O), COR⁴⁰, CR^{39a}R^{39b}, NR⁴⁰, wherein
         m1 is selected from 0, 1 and 2;
         each R^{39a} and R^{39b} is independently selected from halo and C₁₋₄alkyl; and
         R⁴⁰ is selected from hydrogen and C₁₋₄alkyl;
   R³¹ and R^{31a} are independently selected from hydrogen, C₁₋₄alkyl, C₁₋₄ alkoxy, amino, hydroxy, C₃₋₈ cycloalkyl and C₁₋₄ alkyl-amino;
   R³² and R^{32a} are independently selected from halo, carbonyl, C₁₋₄alkyl, C₁₋₄alkoxy, amino, hydroxy, C₃₋₈cycloalkyl and C₁₋₄ alkyl-amino;
   R³³ and R^{33a} are independently selected from hydrogen, halo, carbonyl, C₁₋₄alkyl, Ci.₄alkoxy, amino, hydroxy, C₃₋₈Cycloalkyl and C₁₋₄alkyl-amino;
   R³⁴ and R^{34a} are independently selected from hydrogen, carbonyl, C₁₋₄alkyl, C₁₋₄ alkoxy, amino, hydroxy, C₃₋₈cycloalkyl and C₁₋₄alkyl-amino,
wherein any two groups selected from R³¹, R^{31a}, R³², R^{32a}, R³³, R^{33a}, R³⁴, R^{34a} and R³⁶ can form a 5 to 6 member unsaturated or partially saturated ring;
   R³⁵ is selected from hydrogen, halo, cyano, C₁₋₄ alkyl, C₁₋₄alkoxy, amino-carbonyl, halo-substituted C₁₋₄ alkyl, halo-substituted C₁₋₄ alkoxy, hydroxy-substituted C₁₋₄ alkyl, amino-substituted C₁₋₄alkyl, —S(O)₁₋₂R^{35a}, —C(S)R^{35b}, —C(O)NR^{35a}R^{33b},
and —NR^{35a}C(O)R^{35b}, wherein R^{35a} and R^{35b} are independently selected from hydrogen and C₁₋₄ alkyl;
   R³⁶ and R³⁷ together with the carbon atom to which they are both attached form a 3 to 7 member saturated or partially unsaturated ring that can optionally contain 1 to 3 heteroatoms or groups independently selected from N, C(O), O and S(O)ₘ₁; wherein m1 is selected from 0, 1 and 2; wherein said saturated ring formed by R³⁶ and R³⁷ can be unsubstituted or substituted with 1 to 3 groups independently selected from amino, hydroxy, methoxy, halo, methyl, methyl-amino and isobutyryloxy; and
(ii) a therapeutically effective amount of an EGFR TK inhibitor.

In some embodiments, the has the structure of Formula (IVa), or a pharmaceutically acceptable salt or solvate thereof:

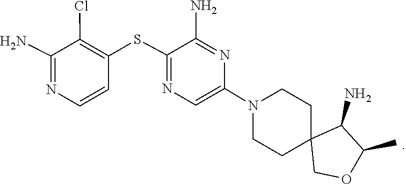 [SMILES: C[C@H]1OCC2(CCN(c3cnc(Sc4ccnc(N)c4Cl)c(N)n3)CC2)[C@H]1N]

In some embodiments, the compound of Formula (IVa) is TNO-155 or a pharmaceutically acceptable salt or solvate thereof. In some embodiments, the SHP2 inhibitor is any of the compounds disclosed in PCT/IB2015/050345 filed Jan. 16, 2015, which is herein incorporated by reference in its entirety.

Any combination of the groups described above for the various variables is contemplated herein. Throughout the specification, groups and substituents thereof are chosen by one skilled in the field to provide stable moieties and compounds.

In some embodiments, the combination comprises a compound as described herein, or a pharmaceutically acceptable salt or solvate thereof (e.g., Formula (Ia)) present at an amount of greater than about 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg. In some embodiments, the compound described herein is present in an amount greater than about 5 mg or about 10 mg. In some embodiments, the composition comprises a compound described herein in an amount from about 1 mg to about 500 mg. In some embodiments, the composition comprises a compound described herein in an amount from about 1 mg to about 10 mg, from about 1 mg to about 25 mg, from about 1 mg to about 50 mg, from about 5 mg to about 10 mg, from about 5 mg to about 25 mg, from about 5 mg to about 50 mg, from about 10 mg to about 25 mg, from about 10 mg to about 50 mg, from about 50 mg to about 100 mg, from about 100 mg to about 200 mg, or from about 200 mg to about 500 mg.

In some embodiments, the combination comprises at least about 1 mg, 2 mg, 3 mg, 4 mg, 5 mg, 10 mg, 15 mg, 20 mg, 25 mg, 30 mg, 35 mg, 40 mg, 45 mg, 50 mg, 60 mg, 70 mg, 80 mg, 85 mg, 90 mg, 100 mg, 125 mg, 150 mg, 175 mg, or 200 mg of the compound described herein, or a pharmaceutically acceptable salt or solvate thereof. In some embodiments, the combination comprises at least about 1 mg, 2 mg, 3 mg, 4 mg, 5 mg, 6 mg, 7 mg, 8 mg, 9 mg, or 10 mg of the compound described herein. In some embodiments, the compound described herein is present in the composition in an amount of at least about 5 mg or about 10 mg. In some embodiments, the combination comprises at least about 1 mg to about 10 mg, about 1 mg to about 25 mg, about 1 mg to about 50 mg, about 5 mg to about 10 mg, about 5 mg to about 25 mg, about 5 mg to about 50 mg, about 10 mg to about 25 mg, about 10 mg to about 50 mg, about 50 mg to about 100 mg, or about 100 mg to about 200 mg of the compound described herein.

In some embodiments, the combination comprises from about 5 mg to about 500 mg or from about 5 mg to about 100 mg of the compound described herein, or a pharmaceutically acceptable salt or solvate thereof. In some embodiments, the combination comprises about 5 mg, about 10 mg, about 20 mg, about 30 mg, about 40 mg, about 50 mg, about 60 mg, about 70 mg, about 80 mg, about 90 mg, about 100 mg, about 125 mg, about 150 mg, about 175 mg, or about 200 mg of the compound described herein.

In some embodiments, the combination comprises a compound described herein, or a pharmaceutically acceptable salt or solvate thereof (e.g., Formula (Ia)) in an amount relative to the weight of the patient (i.e., mg/kg). In some instances, the compound described herein is present in an amount equivalent to about: 0.0001 mg/kg to about 200 mg/kg, 0.001 mg/kg to about 200 mg/kg, 0.01 mg/kg to about 200 mg/kg, 0.01 mg/kg to about 150 mg/kg, 0.01 mg/kg to about 100 mg/kg, 0.01 mg/kg to about 50 mg/kg, 0.01 mg/kg to about 25 mg/kg, 0.01 mg/kg to about 10 mg/kg, or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg, or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg, or 0.5 mg/kg to about 5 mg/kg. In other instances the compound described herein is present in an amount equivalent to about: 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

In some embodiments, the combination comprises from about 5 mg/kg to about 25 mg/kg per patient body weight of a compound described herein, or a pharmaceutically acceptable salt or solvate thereof (e.g., a compound of Formula (Ia)). In some embodiments, the combination comprises about 5 mg/kg, about 10 mg/kg, about 15 mg/kg, about 20 mg/kg, or about 25 mg/kg per body weight of a compound described herein.

In some embodiments, the combination comprises an EGFR TK (epidermal growth factor receptor tyrosine kinase) inhibitor. The epidermal growth factor receptor (EGFR) is involved in the development and progression of cancers. Somatic EGFR mutations are predictors of response to treatment with EGFR tyrosine kinase (TK) inhibitors (TKIs). In some embodiments, an EGFR TK inhibitor can be a small molecule compound, a nucleic acid, a polypeptide, an antibody, a peptibody, a diabody, a minibody, a single-chain variable fragment (ScFv), or a functional fragment or variant thereof. In some embodiments, the EGFR TK inhibitor is a small molecule compound (e.g., a compound having a molecule weight of less than about 1000 Da). In some instances, useful EGFR TK inhibitors in the combinations described herein include nucleic acids and polypeptides. In some embodiments, the EGFR TK inhibitor is a polypeptide (e.g., macrocyclic polypeptide). In some embodiments, the EGFR TK inhibitor is an antibody, peptibody, diabody, minibody, ScFv, or a functional fragment thereof.

In some embodiments, the EGFR TK inhibitor is a small molecule compound. In some embodiments, the EGFR TK inhibitor is selected from erlotinib, afatinib, gefitinib, osimertinib, dacomitinib, icotinib, rociletinib, olmatinib, tarloxotinib, TAK-788, amivantamab (JNJ-6372), or AC0010. In some embodiments, the EGFR TK inhibitor is first generation inhibitor (e.g., erlotinib or gefitinib). In some embodiments, the EGFR TK inhibitor is a second generation inhibitor (e.g., afatinib or Vizimpro (dacomitinib)). In some embodiments, the EGFR TK inhibitor is a third generation inhibitor (e.g., osimertinib, rociletinib, olmatinib, or AC0010).

First-generation EGFR-TKIs function to block the activation of downstream signaling induced by EGFR through binding to the ATP-binding sites. In some embodiments, the EGFR TKI is erlotinib. Erlotinib, sold under the brand name Tarceva among others, and is used to treat some non-small cell lung cancers (NSCLC) with mutations in the epidermal growth factor receptor (EGFR)-either an exon 19 deletion (del19) or exon 21 (L858R) substitution mutation. In some embodiments, the EGFR TKI is gefitinib. Gefitinib, sold under the brand name Iressa, is a medication used for certain breast, lung and other cancers. Gefitinib, like erlotinib, interrupts signaling through the epidermal growth factor receptor (EGFR) in target cell.

In some embodiments, the combination comprises a second generation EGFR TKI. In some embodiments, the EGFR TKI is afatinib. In some embodiments, the EGFR TKI is Vizimpro (Dacomitinib). Dacomitinib, sold under the brand name Vizimpro, is a selective and irreversible inhibitor of EGFR.

In some embodiments, the combination comprises a third generation EGFR TKI. In some embodiments, the EGFR TKI is osimertinib, rociletinib, olmutinib, or AC0010.

In some embodiments, the EGFR TKI is osimertinib. Osimertinib is sold under the name Tagrisso and is often used to treat locally advanced or metastatic cancers that are positive for the T790M mutation. The T790M mutation may be de novo or acquired following first-line treatment with other tyrosine kinase inhibitors (TKIs), such as gefitinib and afatinib.

In some embodiments, the EGFR TKI is olmatinib (HM61713).

In some embodiments, the EGFR TKI is AC0010. AC0010 is a small molecule irreversible tyrosine kinase inhibitor that selectively targets mutant forms of EGFR while sparing wild-type (WT) EGFR.

In some embodiments, the EGFR TKI is tarloxotinib (Tarlox). In some embodiments, the EGFR TKI is TAK-788.

In some embodiments, the EGFR TK inhibitor is an antibody. In some embodiments, the antibody is a monoclonal or polyclonal antibody. In certain embodiments, the antibody is a monoclonal antibody. Antibodies include all known types of antibodies and functional fragments thereof, including but not limited to, those exemplified herein such as, for example, human antibodies, mouse antibodies, chimeric antibodies, humanized antibodies, or chimeric humanized antibodies.

In some embodiments, the EGFR TKI is amivantamab (JNJ-6372). Amivantamab is a fully human EGFR and mesenchymal epithelial transition (MET) bispecific antibody with immune cell-directing activity. Amivantamab targets the Exon 20 mutation which is the third most prevalent EGFR mutation in NSCLC.

The EGFR TK inhibitor can be present in an amount as a measure with regards to the weight of the patient in need thereof. For example, the EGFR TK inhibitor can be present in an amount from about 0.1 mg/kg to about 30 mg/kg, from about 0.1 mg/kg to about 25 mg/kg, from about 0.1 mg/kg to about 20 mg/kg, from about 0.1 mg/kg to about 15 mg/kg, from about 0.1 mg/kg to about 10 mg/kg, from about 0.1 mg/kg to about 7.5 mg/kg, from about 0.1 mg/kg to about 5 mg/kg, from about 0.1 mg/kg to about 2.5 mg/kg, or from about 0.1 mg/kg to about 1 mg/kg. In some embodiments, the EGFR TK inhibitor is present in an amount of about 0.5 mg/kg to about 30 mg/kg, about 0.5 mg/kg to about 25 mg/kg, about 0.5 mg/kg to about 20 mg/kg, about 0.5 mg/kg to about 15 mg/kg, about 0.5 mg/kg to about 10 mg/kg, about 0.5 mg/kg to about 7.5 mg/kg, about 0.5 mg/kg to about 5 mg/kg, about 0.5 mg/kg to about 2.5 mg/kg, or about 0.5 mg/kg to about 1 mg/kg. In some embodiments, the EGFR TK inhibitor is present in an amount of about 0.5 mg/kg to about 5 mg/kg or about 0.1 mg/kg to about 10 mg/kg. In some embodiments, the EGFR TK inhibitor is present in an amount of about 0.5 mg/kg to about 15 mg/kg or about 0.1 mg/kg to about 20 mg/kg.

In some embodiments, the EGFR TK inhibitor is present at an amount of about 0.1 mg/kg, 0.5 mg/kg, 1 mg/kg, 2 mg/kg, 3 mg/kg, 4 mg/kg, 5 mg/kg, 10 mg/kg, 15 mg/kg, 20 mg/kg, or 30 mg/kg. In some embodiments, the EGFR TK inhibitor is present at an amount of about 1 mg/kg, 2 mg/kg, 3 mg/kg, or 5 mg/kg.

In some embodiments, the EGFR TK inhibitor is present in the combination at an amount of about 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. In some embodiments, the EGFR TK inhibitor is present in the combination at an amount of about 1 mg to about 10 mg, about 10 mg to about 20 mg, about 25 mg to about 50 mg, about 30 mg to about 60 mg, about 40 mg to about 50 mg, about 50 mg to about 100 mg, about 75 mg to about 150 mg, about 100 mg to about 200 mg, about 200 mg to about 500 mg, about 500 mg to about 1000 mg, about 1000 mg to about 1200 mg, about 1000 mg to about 1500 mg, about 1200 mg to about 1500 mg, or about 1500 mg to about 2000 mg.

In some embodiments, the EGFR TK inhibitor is present in the combination in an amount of about 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In some embodiments, the EGFR TK inhibitor is present in the combination in an amount of about 1 mg/mL to about 10 mg/mL, about 5 mg/mL to about 10 mg/mL, about 5 mg/mL to about 15 mg/mL, about 10 mg/mL to about 25 mg/mL; about 20 mg/mL to about 30 mg/mL; about 25 mg/mL to about 50 mg/mL, or about 50 mg/mL to about 100 mg/mL.

In some embodiments, the compound as described herein can be provided in amounts that are synergistic with the amount of the EGFR TK inhibitor. The term synergistic refers to a combination described herein (e.g., a compound of Formula (Ia) and an EGFR TK inhibitor, including coadministration with another active agent such as an anti-cancer agent described herein) or a combination of regimens that is more effective than the additive effects of each individual therapy or regimen.

A synergistic effect of a combination described herein can permit the use of lower dosages of one or more of the components of the combination (e.g., a compound of Formula (Ia) or an EGFR TK inhibitor). A synergistic effect can permit less frequent administration of at least one of the administered therapies (e.g., a compound of Formula (Ia) or an EGFR TK inhibitor) to a subject with a disease, disorder, or condition described herein. Such lower dosages and reduced frequency of administration can reduce the toxicity associated with the administration of at least one of the therapies to a subject without reducing the efficacy of the treatment. A synergistic effect avoids or reduces adverse unwanted side effects associated with the use of any therapy.

### Further Forms of Compounds

In some embodiments, a compound disclosed herein possesses one or more stereocenters and each stereocenter exists independently in either the R or S configuration. The compounds presented herein include all diastereomeric, enantiomeric, and epimeric forms as well as the appropriate mixtures thereof. The compounds and methods provided herein include all cis-, trans-, syn-, anti, entgegen (E), and zusammen (Z) isomers as well as the appropriate mixtures thereof. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions,” John Wiley and Sons, Inc., 1981. In one aspect, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. In some embodiments, the design of a prodrug increases the effective water solubility. An example, without limitation, of a prodrug is a compound described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

In one aspect, prodrugs are designed to alter the metabolic stability or the transport characteristics of a drug, to mask side effects or toxicity, to improve the flavor of a drug or to alter other characteristics or properties of a drug. By virtue of knowledge of pharmacokinetic (PK), pharmacodynamic (PD) processes and drug metabolism in vivo, once a pharmaceutically active compound is known, the design of prodrugs of the compound is possible. (see, for example, Nogrady (1985) Medicinal Chemistry A Biochemical Approach, Oxford University Press, New York, pages 388-392; Silverman (1992), The Organic Chemistry of Drug Design and Drug Action, Academic Press, Inc., San Diego, pages 352-401, Rooseboom et al., Pharmacological Reviews, 56:53-102, 2004; Aesop Cho, “Recent Advances in Oral Prodrug Discovery”, Annual Reports in Medicinal Chemistry, Vol. 41, 395-407, 2006; T. Higuchi and V. Stella, Pro-drugs as Novel Delivery Systems, Vol. 14 of the A.C.S. Symposium Series).

In some embodiments, some of the herein-described compounds may be a prodrug for another derivative or active compound.

In some embodiments, sites on the aromatic ring portion of compounds described herein are susceptible to various metabolic reactions Therefore incorporation of appropriate substituents on the aromatic ring structures will reduce, minimize or eliminate this metabolic pathway. In specific embodiments, the appropriate substituent to decrease or eliminate the susceptibility of the aromatic ring to metabolic reactions is, by way of example only, a halogen, or an alkyl group.

In another embodiment, the compounds described herein are labeled isotopically (e.g., with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine, chlorine, and iodine such as, for example, ²H, ³H, ¹³C, ¹⁴C, ¹⁵N, ¹⁸O, ¹⁷O, ³⁵S, ¹⁸F, ³⁶Cl, and ¹²⁵I. In one aspect, isotopically labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements.

In additional or further embodiments, the compounds described herein are metabolized upon administration to an organism in need to produce a metabolite that is then used to produce a desired effect, including a desired therapeutic effect.

“Pharmaceutically acceptable” as used herein, refers a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “pharmaceutically acceptable salt” refers to a salt formulation of a compound that does not cause significant irritation to an organism to which it is administered and does not abrogate the biological activity and properties of the compound. In some embodiments, pharmaceutically acceptable salts are obtained by reacting a compound disclosed herein with an acid to form a salt. Pharmaceutically acceptable salts are also obtained by reacting a compound disclosed herein with a base to form a salt.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g., lithium, sodium, potassium), an alkaline earth ion (e.g., magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexyl-amine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms, particularly solvates. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

### Pharmaceutical Compositions

In one aspect, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

A pharmaceutical composition, as used herein, refers to a mixture of a compound disclosed herein with other chemical components (i.e., pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism.

Pharmaceutical formulations described herein are administrable to a subject in a variety of ways by multiple administration routes, including but not limited to, oral, parenteral (e.g., intravenous, subcutaneous, intramuscular, intramedullary injections, intrathecal, direct intraventricular, intraperitoneal, intralymphatic, intranasal injections), intranasal, buccal, topical or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments, the compounds disclosed herein are administered orally.

In some embodiments, the pharmaceutical formulation is in the form of a tablet. In other embodiments, pharmaceutical formulations of the compounds disclosed herein are in the form of a capsule.

In one aspect, liquid formulation dosage forms for oral administration are in the form of aqueous suspensions or solutions selected from the group including, but not limited to, aqueous oral dispersions, emulsions, solutions, elixirs, gels, and syrups.

For administration by inhalation, a compound disclosed herein is formulated for use as an aerosol, a mist or a powder.

For buccal or sublingual administration, the compositions may take the form of tablets, lozenges, or gels formulated in a conventional manner.

In some embodiments, compounds disclosed herein are prepared as transdermal dosage forms.

In one aspect, a compound disclosed herein is formulated into a pharmaceutical composition suitable for intramuscular, subcutaneous, or intravenous injection.

In some embodiments, the compound disclosed herein is be administered topically and can be formulated into a variety of topically administrable compositions, such as solutions, suspensions, lotions, gels, pastes, medicated sticks, balms, creams or ointments.

In some embodiments, the compounds disclosed herein are formulated in rectal compositions such as enemas, rectal gels, rectal foams, rectal aerosols, suppositories, jelly suppositories, or retention enemas.

Pharmaceutical compositions and dosage forms described herein typically include one or more excipients. Suitable excipients are well known to those skilled in the art of pharmacy. Whether a certain excipient is suitable for incorporation into a pharmaceutical composition or dosage form depends on a variety of factors such as, for example, the intended route of administration to the patient. Pharmaceutical compositions described herein can include other agents such as stabilizers, lubricants, buffers, and disintegrants that can reduce the rate by which an active ingredient can decompose in a certain formulation.

Pharmaceutical compositions described herein can in certain instances include additional active agents other than those in the combinations described herein (e.g., an anti-cancer agent such as those described herein) in an amount provided herein.

In some embodiments, the compounds described herein are provided in an oral dosage form such as a tablet or capsule. In some embodiment, the compounds described herein are supplied as a powder (e.g., lyophilized powder) that can be resuspended in a liquid suitable for parenteral administration.

EGFR TK inhibitors described herein can be provided in forms convenient to or facilitate their administration to a patient. For example, in some embodiments, the EGFR TK inhibitor can be formulated for oral administration as a table, capsule or pill. In other examples, the EGFR TK inhibitor can be formulated as a ready to use solution for parenteral administration. In some embodiments, the EGFR TK inhibitor, can be formulated as a powder (e.g., lyophilized powder) that can be resuspended in a liquid suitable for parenteral administration. In some embodiment, the combination includes an EGFR TK inhibitor formulated for intravenous administration.

In some embodiments, the combination includes a compound as described herein is formulated as an oral dosage form (e.g., a tablet or capsule) and an EGFR TK inhibitor formulated as an oral dosage form (e.g., a tablet or capsule).

Combinations described herein can be provided as controlled release pharmaceutical products, which have a goal of improving drug therapy over that achieved by their non-controlled counterparts. Controlled release formulations can extend activity of the drug, reduce dosage frequency, and increase subject compliance. In addition, controlled release formulations can be used to affect the time of onset of action or other characteristics, such as blood levels of the drug, and can thus affect the occurrence of side (e.g., adverse) effects

### Methods of Treatment

The combinations and pharmaceutical compositions described herein are useful for treating diseases, disorders, or alleviating or eliminating the symptoms of diseases and disorders such as, for example, cancer.

In an aspect, described herein is a method of treating cancer in a patient in need thereof, the method comprising administering to the patient a combination comprising a SHP2 inhibitor compound as described herein, or a pharmaceutically acceptable salt or solvate thereof (e.g., a compound of Formula (Ia)), and an EGFR TK inhibitor.

In some embodiments, the cancer is in the form of a tumor. In some embodiments, the cancer is selected from squamous cell carcinoma, non-squamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST). In some embodiments, the cancer is squamous cell carcinoma. In some embodiments, the cancer is non-squamous cell carcinoma. In some embodiments, the cancer is non-small cell lung cancer (NSCLC). In some embodiments, the cancer is small cell lung cancer. In some embodiments, the cancer is melanoma. In some embodiments, the cancer is hepatocellular carcinoma. In some embodiments, the cancer is renal cell carcinoma. In some embodiments, the cancer is ovarian cancer. In some embodiments, the caner is head and neck cancer. In some embodiments, the cancer is urothelial cancer. In some embodiments, the cancer is breast cancer (e.g., HER2 negative or HER2 positive breast cancer). In some embodiments, the cancer is prostate cancer. In some embodiments, the cancer is glioblastoma. In some embodiments, the cancer is colorectal cancer. In some embodiments, the care is pancreatic cancer. In some embodiments, the cancer is lymphoma. In some embodiments, the cancer is synovial sarcoma. In some embodiments, the cancer is malignant peripheral sheath tumor (MPNST).

In some embodiments, the tumor is a solid tumor. In some embodiments, the method of treating cancer reduces the tumor volume or tumor burden in the patient. In some embodiments, the tumor is reduced in volume from 5% to 95% or 5% to 50% or any value therein. In some embodiments, the tumor is reduced in volume by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the tumor is reduced in volume by at least 5%, at least 10%, at least 20%, at least 30%, at least 40%, or at least 50%. In some embodiments, the tumor is reduced by about 10% to about 99%. In some embodiments, the tumor is reduced by about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99%. In some embodiments, the tumor is reduced by about 10%, about 20%, about 30%, about 40%, about 50%, about 60%, about 70%, about 80%, about 90%, or about 99%. In some embodiments, the tumor is reduced by at least about 10%, about 20%, about 30%, about 40%, about 50%, about 60%, about 70%, about 80%, or about 90%. In some embodiments, the tumor is reduced by at most about 20%, about 30%, about 40%, about 50%, about 60%, about 70%, about 80%, about 90%, or about 99%.

In some embodiments, the cancer is a hematological cancer. In some embodiments, the cancer is a hematological cancer selected from lymphoma, Non-Hodgkin's lymphoma (NHL), Hodgkin's Lymphoma, Reed-Sternberg disease, multiple myeloma (MM), acute myelogenous leukemia (AML), chronic myelogenous leukemia (CML), acute lymphocytic leukemia (ALL), or chronic lymphocytic leukemia (CLL). In some embodiments, the cancer is Hodgkin's Lymphoma or Reed-Sternberg disease.

In some embodiments, the cancer is lymphoma. In some embodiments, the cancer is Non-Hodgkin lymphoma (NHL). In some embodiments, the NHL is indolent NHL (e.g., follicular lymphoma (FL); lymphoplasmacytic lymphoma (LL); marginal zone lymphoma (MZL) or primary cutaneous anaplastic large cell lymphoma) or aggressive NHL (e.g., Diffuse large B-cell lymphoma (DLBCL); Follicular large cell lymphoma stage III; anaplastic large cell lymphoma; extranodal NK-/T-cell lymphoma; lymphomatoid granulmatosis; angioimmunoblastic T-cell lymphoma; peripheral T-cell lymphoma; intravascular large B-cell lymphoma; Burkitt lymphoma; lymphoblastic lymphoma; adult T-cell leukemia/lymphoma; or mantle cell lymphoma). In some embodiments, the cancer is Hodgkin's lymphoma (e.g., classical or nodular lymphocyte-predominant). In some embodiments, the Hodgkin's Lymphoma includes Reed-Sternberg cells and can cause Reed-Sternberg disease. In some embodiments, the cancer is multiple myeloma (MM). In some embodiments, the cancer is acute myelogenous leukemia (AML). In some embodiments, the cancer is chronic myelogenous leukemia (CML). In some embodiments, the cancer is chronic lymphocytic leukemia (CLL), (e.g., Binet Stage A cancer or a Binet Stage B cancer). In some embodiments, the cancer is acute lymphocytic leukemia (ALL), (e.g., T-cell or B-cell lymphoblastic leukemia).

In some embodiments, the cancer is a Stage I, Stage II, Stage, III, or Stage IV cancer. In some embodiments, the cancer is a Stage I cancer (e.g., Stage IA, IB, or IC). In some embodiments, the cancer is a Stage II cancer (e.g., Stage IIA or IIB). In some embodiments, the cancer is a Stage III cancer, (e.g., Stage IIIA, IIIB, or IIIC). In some embodiments, the cancer is a Stage IV cancer, (e.g., Stage IVA or IVB).

The combinations described herein can be administered to a cancer patient at any time following diagnosis. For example, the cancer patient can be treatment naive (i.e., has not received a cancer therapy for the diagnosed cancer). The cancer patient can be treatment naive for one cancer but can be diagnosed with one or more other cancers resulting from, for example, metastasis or malignancy. The cancer patient can be immune checkpoint naive for one or more cancers. The cancer patient can have a cancer that is refractory. In certain instances, the combinations described herein are administered as a first line therapy (e.g., the first therapy administered to a treatment naive cancer patient) to a patient in need thereof.

In some embodiments, the method of treating cancer inhibits metastasis of the cancer in the patient. In some embodiments, metastasis is inhibited by at least about ₅%, 10%, 20%, 30%, 40%, 50%, 60%, 70%, 80%, 90%, or 100%.

In some embodiments, the method of treating cancer reduces pre-existing tumor metastasis in the patient. In some embodiments, preexisting tumor metastasis is reduced by at least about 5%, 10%, 20%, 30%, 40%, 50%, 60%, 70%, 80%, 90%, or 100%.

In some embodiments, the method of treating cancer prolongs or increases the time to disease progression of the cancer in the patient (including progression between advanced stages; e.g., progression from Stage III to Stage IV cancer). In some embodiments, the increase is a comparison between the time to disease progression with and without treatment. In some embodiments, the methods described herein prolong the time to disease progression by at least 1 week, 2 weeks, 3 weeks, 4 weeks, 1 month, 2 months, 3 months, 4 months, 5 months, 6 months, 7 months, 8 months, 9 months, 10 months, 11 months, 1 year, or more, including values therein.

In some embodiments, the method of treating cancer prolongs the survival of the patient. In some embodiments, the method of treating cancer increases progression-free survival of the patient. In some embodiments, the method of treating cancer prolongs the time to disease progression of the cancer in the patient. In some embodiments, the method of treating cancer prolongs the survival of the patient. In some embodiments, the method of treating cancer increases progression-free survival of the patient. In some embodiments, survival is prolonged by at least 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years, or more, including values therein.

In some embodiments, the patient is treatment naïve patient is treatment naïve.

In some embodiments, the method comprises administering the combinations described herein to the patient as a first line therapy. In some embodiments, the method comprises administering the combination described herein to the patient as a second, third, fourth, fifth, or sixth line of treatment. In some embodiments, the method comprises administering the combination described herein as a second line of treatment. In some embodiments, the method comprises administering the combination described herein as a third line of treatment.

In some embodiments, the method comprises administering the combinations described herein to the patient following treatment with at least one anti-cancer therapy. In some embodiments, the anti-cancer therapy is chemotherapy, radiotherapy, surgery, targeted therapy, immunotherapy, or a combination thereof. In some embodiments, the anti-cancer therapy is chemotherapy. In some embodiments, the anti-cancer therapy is radiotherapy. In some embodiments, the anti-cancer therapy is cancer surgery. In some embodiments, the anti-cancer therapy is tumor resection or excision. In some embodiments, the anti-cancer therapy is immunotherapy.

In some embodiments, the method comprises administering the combination described herein to a patient who has failed at least one EGFR TK inhibitor therapy. In some embodiments, the method comprises administering the combination described herein to a patient who has failed at least one osimertinib therapy. In some embodiments, the method comprises administering the combination described herein to a patient afflicted by lung cancer. In some embodiments, the method comprises administering the combination described herein to a patient who is afflicted with a cancer comprising NCI-H1975 cancer cells.

In some embodiments, the cancer is resistant to at least one anti-cancer agent

### Methods of Dosing and Treatment Regimens

In another aspect, the combination described herein are used in the preparation of medicaments for the treatment of diseases or conditions described herein.

In certain embodiments, the combination disclosed herein are administered for prophylactic and/or therapeutic treatments. In certain therapeutic applications, the combinations are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation clinical trial.

In prophylactic applications, the combinations disclosed herein are administered to a patient susceptible to or otherwise at risk of a particular disease, disorder or condition.

In some embodiments, the method comprises administering the combinations described herein to the patient orally or by intraperitoneal methods (i.p.) or a combination thereof. In some embodiments, the combination is administered orally. In some embodiments, the combination is administered orally. In some embodiments, the combination is administered by i.p, methods. In some embodiments, the combination is administered intravenously (I.V.).

Doses employed for adult human treatment are typically in the range of 0.01 mg-5000 mg per day or from about 0.01 mg to about 1000 mg per day. In one embodiment, the desired dose is conveniently presented in a single dose or in divided doses.

In certain embodiments, the dose of drug being administered may be temporarily reduced or temporarily suspended for a certain length of time (i.e., a “drug holiday”).

In some embodiments, the method comprises administering the combinations described herein to the patient daily, weekly, or monthly. In some embodiments, the combination is administered daily. In some embodiments, the combination is administered weekly. In some embodiments, the combination is administered bi-weekly. In some embodiments, the combination is administered monthly. In some embodiments, the combination is administered bi-monthly.

The combinations described herein can be administered, for example, once a day (QD), twice daily (BID), once a week (QW), twice weekly (BID), three times a week (TIW), or monthly (QM). In some embodiments, the method comprises administering the combinations described herein QD, BID, or TID. In some embodiments, the combination is administered QD. In some embodiments, the combination is administered BID. In some embodiments, the combination is administered TID. In certain instances, the compound described herein is administered 2 to 3 times a week. In another embodiment, the compound described herein is administered QD. The compound can be administered QD for about: 1 day to about 7 days, 1 day to about 14 days, 1 day to about 21 days, 1 day to about 28 days, or daily until disease progression or unacceptable toxicity. The administration of a compound described herein can, in part, depend upon the tolerance of the patient where greater tolerance can allow greater or more frequent administration.

The term “administered simultaneously”, as used herein, is not specifically restricted and means that the compounds of the present disclosure and the additional active agent are substantially administered at the same time, e.g., as a mixture or in immediate subsequent sequence.

The term “administered sequentially”, as used herein, is not specifically restricted and means that the compounds of the present disclosure and the additional active agent are not administered at the same time but one after the other, or in groups, with a specific time interval between administrations. The time interval may be the same or different between the respective administrations of the compounds of the present disclosure and the additional active agent and may be selected, for example, from the range of 2 minutes to 96 hours, 1 to 7 days or one, two, or three weeks. Generally, the time interval between the administrations may be in the range of a few minutes to hours, such as in the range of 2 minutes to 72 hours, 30 minutes to 24 hours, or 1 to 12 hours. Further examples include time intervals in the range of 24 to 96 hours, 12 to 36 hours, 8 to 24 hours, and 6 to 12 hours.

In some embodiments, the SHP2 inhibitor compound described herein, or a pharmaceutically acceptable salt or solvate thereof and the EGFR TK inhibitor are administered concurrently or sequentially. In some embodiments, the SHP2 inhibitor compound described herein and the EGFR TK inhibitor are administered sequentially. In some embodiments, the SHP2 inhibitor described herein, or a pharmaceutically acceptable salt or solvate thereof (e.g., a compound of Formula (Ia)), is administered QD, BID, or TID; and the EGFR TK inhibitor is administered QD, BID, or TID.

The combinations described herein can include administration of each therapy (e.g., a compound of Formula (Ia) and an EGFR TK inhibitor), where the administration is performed simultaneously or sequentially (in either order). In some embodiments, the SHP2 inhibitor compound described herein and the EGFR TK inhibitor are administered simultaneously (e.g., within at least 1 to 5 min of each other). In other embodiments, the compound of Formula (I) and the EGFR TK inhibitor are administered sequentially (e.g., within at least 10 min, 15 min, 30 min, 1 h, 2 h, 5 h, 10 h, 12 h, 1 day, 2 days, 5 days, 7 days, 14 days, or 21 days of each other).

In some embodiments, the SHP2 inhibitor compound described herein is administered concurrently with an EGFR TK inhibitor. In some embodiments, the compound described herein is administered prior to an EGFR TK inhibitor. In some embodiments, the compound described herein is administered after an EGFR TK inhibitor.

The combinations described herein can be administered in a regimen. The regimen can be structured to provide therapeutically effective amounts of a SHP2 inhibitor compound described herein and an EGFR TK inhibitor over a predetermined period of time (e.g., an administration time). The regimen can be structured to limit or prevent side-effects or undesired complications of each of the components of the combination described herein. The regimen can be structured in a manner that results in increased effect for both therapies of the combination (e.g., synergy). Regimens useful for treating cancer can include any number of days of administration which can be repeated as necessary. Administration periods can be broken by a rest period that includes no administration of at least one therapy. For example, a regimen can include administration periods that include 2, 3, 5, 7, 10, 15, 21, 28, or more days. These periods can be repeated. For example, a regimen can include a set number of days as previously described where the regimen is repeated 1, 2, 3, 4, 5, 6, 7, 8, 9, 10, 11, 12, 13, or more times.

Regimens can include a rest period of at least 1, 2, 3, 5, 7, 10, or more days, where at least one therapy is no longer administered to a patient. The rest period can be determined by, for example, monitoring the reaction of the patient to the drug or by measuring the efficacy of the treatment. A rest period can be applicable to a single therapy, such that only one therapy of a combination described herein is discontinued in the rest period but the other therapy or therapies are still administered. Rest periods can be applied to all of the therapies administered to the subject such that the subject receives no therapy for a set period of time during the rest period.

Regimens described herein for the treatment of cancer using the combinations described herein can be continued until disease progression or unacceptable toxicity.

### Biomarkers

In another aspect, presented herein is a method of modulating one or more biomarkers over baseline levels prior to treatment in a patient in need thereof, comprising administering to the patient a combination of a SHP2 inhibitor compound described herein, or a pharmaceutically acceptable salt or solvate thereof, (e.g., a compound of Formula (Ia)); and an EGFR TK inhibitor.

In some embodiments, the one or more biomarkers in increased or decreased over baseline levels prior to treatment. In some embodiments, the one or more biomarkers is increased over baseline levels. In some embodiments, the one or more biomarkers is decreased over baseline levels.

In some embodiments, the one or more biomarkers is increased by at least 5%, at least 10%, at least 15%, at least 20%, at least 25%, at least 30%, at least 40%, at least 50%, at least 100%, or at least 150%. In some embodiments, the one or more biomarkers is increased by at least 1.5 times, 2 times, 3 times, 4 times, 5 times, 10 times, 15 times, 20 times, or 25 times. In some embodiments, the one or more biomarkers is decreased by at least 5%, at least 10%, at least 15%, at least 20%, at least 25%, at least 30%, at least 40%, at least 50%, at least 100%, or at least 150%. In some embodiments, the one or more biomarkers is decreased by at least 1.5 times, 2 times, 3 times, 4 times, 5 times, 10 times, 15 times, 20 times, or 25 times.

### DEFINITIONS

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed invention.

The terms below, as used herein, have the following meanings, unless indicated otherwise:

“Oxo” refers to the ═O substituent.

“Alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₈ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (i-propyl), n-butyl, i-butyl, s-butyl, n-pentyl, 1,1-dimethylethyl (t-butyl), 3-methylhexyl, 2-methylhexyl, 1-ethyl-propyl, and the like. In some embodiments, the alkyl is methyl or ethyl. Preferably, the C₁-C₁₀ alkyl is any one of methyl, ethyl, n-propyl, isopropyl, and tert-butyl. Unless stated otherwise specifically in the specification, an alkyl group may be optionally substituted as described below.

“Alkylene” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—.

“Alkoxy” refers to a radical of the formula-OR where R is an alkyl radical as defined. Unless stated otherwise specifically in the specification, an alkoxy group may be optionally substituted as described below. Representative alkoxy groups include, but are not limited to, methoxy, ethoxy, propoxy, butoxy, pentoxy. In some embodiments, the alkoxy is methoxy. In some embodiments, the alkoxy is ethoxy. The term “C₁-C₁₀ alkoxy” alone or in combination means the group C₁-C₁₀ alkyl-O—, wherein “C₁-C₁₀ alkyl” means as defined above, which includes, but not limited to, methoxy (—OCH₃), ethoxy (—OCH₂CH₃), n-propoxy (—OCH₂CH₂CH₃), iso-propoxy (—OCH(CH₃)₂), n-butoxy (—OCH₂CH₂CH₂CH₃), sec-butoxy (—OCH(CH₃)CH₂CH₃), iso-butoxy (—OCH₂CH(CH₃)₂), tert-butoxy (—OC(CH₃)₃), etc.

“Heteroalkyl” refers to an alkyl radical as described above where one or more carbon atoms of the alkyl is replaced with a O, N (i.e., NH, N-alkyl) or S atom. “Heteroalkylene” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkyl groups include, but are not limited to —OCH₂OMe, —OCH₂CH₂OMe, or —OCH₂CH₂OCH₂CH₂NH₂. Representative heteroalkylene groups include, but are not limited to —OCH₂CH₂O—, —OCH₂CH₂OCH₂CH₂O—, or —OCH₂CH₂OCH₂CH₂OCH₂CH₂O—.

“Alkylamino” refers to a radical of the formula —NHR or —NRR where each R is, independently, an alkyl radical as defined above. Unless stated otherwise specifically in the specification, an alkylamino group may be optionally substituted as described below.

The term “aromatic” refers to a planar ring having a delocalized π-electron system containing 4n+2 π electrons, where n is an integer. Aromatics can be optionally substituted. The term “aromatic” includes both aryl groups (e.g., phenyl, naphthyl) and heteroaryl groups (e.g., pyridinyl, quinolinyl).

“Aryl” refers to an aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted.

“Carboxy” refers to —CO₂H. In some embodiments, carboxy moieties may be replaced with a “carboxylic acid bioisostere”, which refers to a functional group or moiety that exhibits similar physical and/or chemical properties as a carboxylic acid moiety. A carboxylic acid bioisostere has similar biological properties to that of a carboxylic acid group. A compound with a carboxylic acid moiety can have the carboxylic acid moiety exchanged with a carboxylic acid bioisostere and have similar physical and/or biological properties when compared to the carboxylic acid-containing compound. For example, in one embodiment, a carboxylic acid bioisostere would ionize at physiological pH to roughly the same extent as a carboxylic acid group. Examples of bioisosteres of a carboxylic acid include, but are not limited to:

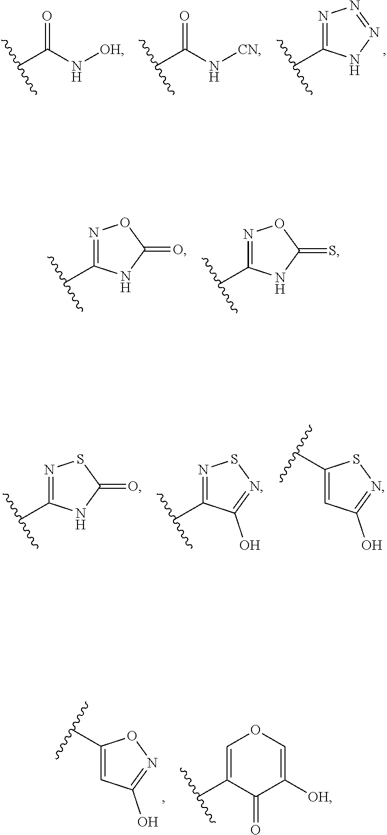

and the like.

“Cycloalkyl” refers to a monocyclic or polycyclic non-aromatic radical, wherein each of the atoms forming the ring (i.e., skeletal atoms) is a carbon atom. Cycloalkyls may be saturated, or partially unsaturated. Cycloalkyls may be fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. In some embodiments, a cycloalkyl is a C₃-C₆cycloalkyl. In some embodiments, the cycloalkyl is monocyclic, bicyclic or polycyclic. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, decalinyl and adamantyl. In some embodiments, the cycloalkyl is monocyclic. Monocyclic cyclcoalkyl radicals include, for example, cyclopropyl, cyclobutyl, cyclopentyl, cyclohexyl, cycloheptyl, and cyclooctyl. In some embodiments, the monocyclic cyclcoalkyl is cyclopropyl, cyclobutyl, cyclopentyl or cyclohexyl. In some embodiments, the cycloalkyl is bicyclic. Bicyclic cycloalkyl groups include fused bicyclic cycloalkyl groups, spiro bicyclic cycloalkyl groups, and bridged bicyclic cycloalkyl groups. In some embodiments, cycloalkyl groups are selected from among spiro[2.2]pentyl, bicyclo[1.1.1]pentyl, bicyclo[3.3.0]octane, bicyclo[4.3.0]nonane, bicyclo[2.1.1]hexane, bicyclo[2.2.1]heptane, bicyclo[2.2.2]octane, bicyclo[3.2.2]nonane, bicyclo[3.3.2]decane, norbornyl, 3,4-dihydronaphthalen-1 (2H)-one and decalinyl. In some embodiments, the cycloalkyl is polycyclic. Polycyclic radicals include, for example, adamantyl, and. In some embodiments, the polycyclic cycloalkyl is adamantyl. Unless otherwise stated specifically in the specification, a cycloalkyl group may be optionally substituted.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” refers to an alkoxy radical, as defined above, that is substituted by one or more halo radicals, as defined above, e.g., trifluoromethoxy, difluoromethoxy, fluoromethoxy, trichloromethoxy, 2,2,2-trifluoroethoxy, 1,2-difluoroethoxy, 3-bromo-2-fluoropropoxy, 1,2-dibromoethoxy, and the like. Unless stated otherwise specifically in the specification, a haloalkoxy group may be optionally substituted.

“Heterocycloalkyl” or “heterocyclyl” or “heterocyclic ring” refers to a stable 3- to 14-membered non-aromatic ring radical comprising 2 to 10 carbon atoms and from one to 4 heteroatoms selected from the group consisting of nitrogen, oxygen, and sulfur. Unless stated otherwise specifically in the specification, the heterocycloalkyl radical may be a monocyclic, bicyclic ring (which may include a fused bicyclic heterocycloalkyl (when fused with an aryl or a heteroaryl ring, the heterocycloalkyl is bonded through a non-aromatic ring atom), bridged heterocycloalkyl or spiro-heterocycloalkyl), or polycyclic. In some embodiments, the heterocycloalkyl is monocyclic or bicyclic. In some embodiments, the heterocycloalkyl is monocyclic. In some embodiments, the heterocycloalkyl is bicyclic. The nitrogen, carbon or sulfur atoms in the heterocyclyl radical may be optionally oxidized. The nitrogen atom may be optionally quaternized. The heterocycloalkyl radical is partially or fully saturated. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 10 carbons in the ring. In some embodiments, heterocycloalkyls have from 2 to 8 carbons in the ring. In some embodiments, heterocycloalkyls have from 2 to 8 carbons in the ring and 1 or 2 N atoms. In some embodiments, heterocycloalkyls have from 2 to 10 carbons, 0-2 N atoms, 0-2 O atoms, and 0-1 S atoms in the ring. In some embodiments, heterocycloalkyls have from 2 to 10 carbons, 1-2 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). Unless stated otherwise specifically in the specification, a heterocycloalkyl group may be optionally substituted. In some embodiments, the term “3-12 membered heterocyclic group” refers to a saturated or partially unsaturated monocyclic ring containing 3-12, particularly 5-12, more particularly 5-7 carbon atoms and heteroatoms or heteroatom groups or a polycyclic heterocyclic group, the heteroatom or heteroatom group is selected from N, NH, O, C(O), S(O)ₘ (where m is 0, 1 or 2). In some embodiments, the 3-12-membered heterocyclic groups include aziridinyl, azetidinyl, oxetanyl, pyrrolidinyl, tetrahydrofuranyl, tetrahydrothienyl, piperidinyl, morpholinyl, piperazinyl, thiomorpholine, tetrahydropyranyl, 1,1-dioxothiomorpholinyl, butyrolactamyl, valerolactam, caprolactam, butyrolactone, valerolactone, or caprolactone.

“Heteroaryl” refers to an aryl group that includes one or more ring heteroatoms selected from nitrogen, oxygen and sulfur. The heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-4 N atoms in the ring. In some embodiments, a heteroaryl contains 1~4 N atoms in the ring. In some embodiments, a heteroaryl contains 0-4 N atoms, 0-1 O atoms, and 0-1 S atoms in the ring. In some embodiments, a heteroaryl contains 1~4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, a bicyclic heteroaryl is a C₆-C₉heteroaryl.

The term “optionally substituted” or “substituted” means that the referenced group may be substituted with one or more additional group(s) individually and independently selected from alkyl, haloalkyl, cycloalkyl, aryl, heteroaryl, heterocycloalkyl, —OH, alkoxy, aryloxy, alkylthio, arylthio, alkylsulfoxide, arylsulfoxide, alkylsulfone, arylsulfone, —CN, alkyne, C₁-C₆alkylalkyne, halogen, acyl, acyloxy, —CO₂H, —CO₂alkyl, nitro, and amino, including mono- and di-substituted amino groups (e.g., —NH₂, —NHR, —NR₂), and the protected derivatives thereof. In some embodiments, optional substituents are independently selected from alkyl, alkoxy, haloalkyl, cycloalkyl, halogen, —CN, —NH₂, —NH(CH₃), —N(CH₃)₂, —OH, —CO₂H, and —CO₂alkyl. In some embodiments, optional substituents are independently selected from fluoro, chloro, bromo, iodo, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

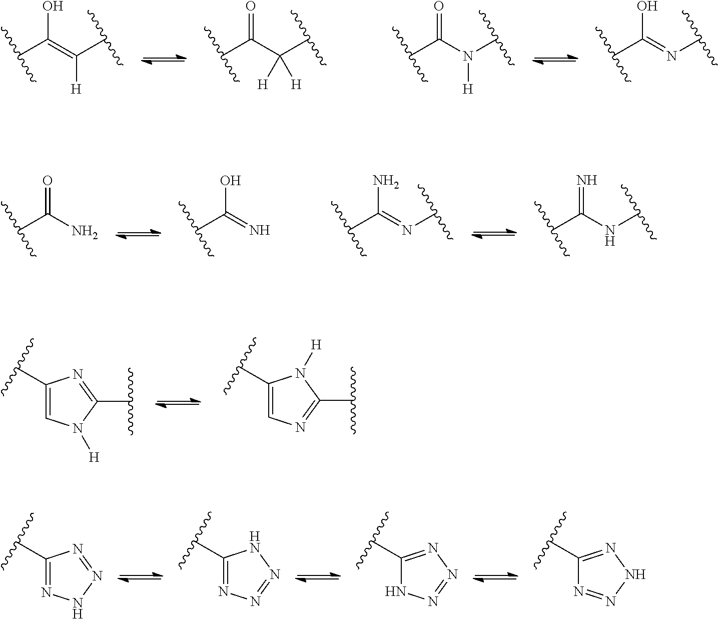

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. An “effective amount” is an amount sufficient for a compound to accomplish a stated purpose relative to the absence of the compound (e.g., achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “pharmaceutical combination” as used herein, means a product that results from the mixing or combining of more than one active ingredient and includes both fixed and non-fixed combinations of the active ingredients. The term “fixed combination” means that the active ingredients, e.g., a compound of Formula (Ia) and a co-agent, are both administered to a patient simultaneously in the form of a single entity or dosage. The term “non-fixed combination” means that the active ingredients, e.g., a compound of Formula (Ia) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g., the administration of three or more active ingredients.

The term “subject” or “patient” encompasses mammals. Examples of mammals include, but are not limited to, humans. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

The term “cancer” refers to any physiological condition in mammals characterized by unregulated cell growth. Cancers described herein include solid tumors and hematological (blood) cancers. A “hematological cancer” refers to any blood borne cancer and includes, for example, myelomas, lymphomas and leukemias. A “solid tumor” or “tumor” refers to a lesion and neoplastic cell growth and proliferation, whether malignant or benign, and all pre-cancerous and cancerous cells and tissues resulting in abnormal tissue growth. “Neoplastic,” as used herein, refers to any form of dysregulated or unregulated cell growth, whether malignant or benign, resulting in abnormal tissue growth.

The term “enhance” refers to an increase or improvement in the function or activity of a protein or cell after administration or contacting with a combination described herein compared to the protein or cell prior to such administration or contact.

The term “anti-cancer agent” is used in accordance with its plain ordinary meaning and refers to a composition having anti-neoplastic properties or the ability to inhibit the growth or proliferation of cells. In embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer.

The term “chemotherapeutic” or “chemotherapeutic agent” is used in accordance with its plain ordinary meaning and refers to a chemical composition or compound having anti-neoplastic properties or the ability to inhibit the growth or proliferation of cells. “Chemotherapy” refers to a therapy or regimen that includes administration of a chemotherapeutic or anti-cancer agent described herein.

The terms “polypeptide” and “protein” are used interchangeably herein and refer to any molecule that includes at least 2 or more amino acids.

The term “regimen” refers to a protocol for dosing and timing the administration of one or more therapies (e.g., combinations described herein or another active agent such as for example an anti-cancer agent described herein) for treating a disease, disorder, or condition described herein. A regimen can include periods of active administration and periods of rest as known in the art.

Antibodies described herein can be polyclonal or monoclonal and include xenogeneic, allogeneic, or syngeneic forms and modified versions thereof (e.g., humanized or chimeric). An “antibody” is intended to mean a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (Borrebaeck (ed.) (1995) Antibody Engineering, Second Edition, Oxford University Press; Kuby (1997) Immunology, Third Edition, W.H. Freeman and Company, New York). Specific molecular antigens that can be bound by an antibody described herein include EGFR tyrosine kinase (TK) and its epitopes.

The term “monoclonal antibody (ies)” refers to a population of antibody molecules that contain one species of an antigen binding site capable of immunoreacting with a particular epitope of an antigen, whereas the term “polyclonal antibody (ies)” refers to a population of antibody molecules that contain multiple species of antigen binding sites capable of interacting with a particular antigen. A monoclonal antibody, typically displays a single binding affinity for a particular antigen with which it immuno-reacts. For example, the monoclonal antibodies to be used in accordance with the present invention can be made by a variety of techniques, including, for example, the hybridoma method (e.g., Kohler and Milstein., Nature, 256:495-97 (1975); Hongo et al., Hybridoma, 14 (3): 253-260 (1995), Harlow et al., Antibodies: A Laboratory Manual, (Cold Spring Harbor Laboratory Press, 2^{nd} ed. 1988); Hammerling et al., Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352:624-628 (1991); Marks et al., J Mol. Biol. 222:581-597 (1992); Sidhu et al., Mol. Biol. 338 (2): 299-310 (2004); Lee et al., Mol. Biol. 340 (5): 1073-1093 (2004); Fellouse, Proc. Natl. Acad. Set USA 101 (34): 12467-12472 (2004); and Lee et al., Immunol. Methods 284 (1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Set USA 90:2551 (1993); Jakobovits et al., Nature 362:255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10:779-783 (1992); Lon berg et al., Nature 368:856-859 (1994); Morrison, Nature 368:812-813 (1994); Fishwild et al., Nature Biotechnol. 14:845-851 (1996); Neuberger, Nature Biotechnol. 14:826 (1996); and Lonberg and Huszar, Intern. Rev. Immunol. 13:65-93 (1995).

The monoclonal antibodies herein also include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Set USA, pp. 6851-6855 (1984)). “Humanized antibody(ies)” can be considered as a subset of chimeric antibodies described herein.

The term “human” when used in reference to an antibody or a functional fragment thereof (e.g., “humanized antibody(ies))” refers an antibody or functional fragment thereof that has a human variable region or a portion thereof corresponding to human germline immunoglobulin sequences. Such human germline immunoglobulin sequences are described by Kabat et al. (1991) Sequences of Proteins of Immunological Interest, Fifth Edition, U.S. Department of Health and Human Services, NIH Publication No. 91-3242. In certain embodiments, a human antibody is an antibody that possesses an amino acid sequence corresponding to that of an antibody produced by a human and/or has been made using any of the techniques for making human antibodies as disclosed herein. Human antibodies can be produced using various techniques known in the art, including phage-display libraries. Hoogenboom and Winter, Mol. Biol., 227:381 (1991); Marks et al., Mol. Biol., 222:581 (1991). Also available for the preparation of human monoclonal antibodies are methods described in Cole et al., Monoclonal Antibodies and Cancer Therapy, Alan R. Liss, p. 77 (1985); Boemer et al., Immunol., 147 (1): 86-95 (1991). See also van Dijk and van de Winkel, Curr. Opin. Pharmacol., 2:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE technology). See also, for example, Li et al., Proc. Natl. Acad. Sci. USA, 103:3557-3562 (2006) regarding human antibodies generated via a human B-cell hybridoma technology.

A “humanized antibody” refers to antibodies made by a non-human cell having variable or variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention can include amino acid residues not encoded by human germline immunoglobulin sequences (e.g., mutations introduced by random or site-specific mutagenesis in vitro or by somatic mutation in vivo), for example in the CDRs. Humanized antibodies can also include antibodies in which CDR sequences derived from the germline of another mammalian species, such as a mouse, have been grafted onto human framework sequences.

Humanized forms of non-human (e.g., murine) antibodies are antibodies that contain minimal sequence derived from non-human immunoglobulin. In some embodiments, a humanized antibody is a human immunoglobulin (recipient antibody) in which residues from a hypervariable of the recipient are replaced by residues from an hypervariable region of a nonhuman species (donor antibody) such as mouse, rat, rabbit or non-human primate having the desired specificity, affinity, and/or capacity. In some instances, framework (“FR”) residues of the human immunoglobulin are replaced by corresponding non-human residues. Furthermore, humanized antibodies can comprise residues that are not found in the recipient antibody or in the donor antibody. These modifications can be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions can include one or more individual FR residue substitutions that improve antibody performance, such as binding affinity, isomerization, immunogenicity, etc. The number of these amino acid substitutions in the FR are typically no more than 6 in the H chain, and in the L chain, no more than 3. The humanized antibody optionally can also include at least a portion of an immunoglobulin constant region (Fc), which can be a human immunoglobulin. Exemplary methods and humanized antibodies include those described by Jones et al. Nature 321:522-525 (1986); Riechmann et al. Nature 332:323-329 (1988); and Presta, Curr. Op. Struct. Biol. 2:593-596 (1992); Vaswani and Hamilton, Ann. Allergy. Asthma & Immunol. 1:105-115 (1998); Harris, Biochem. Soc. Transactions 23:1035-1038 (1995); Burle and Gross, Curr. Op. Biotech. 5:428-433 (1994); and U.S. Pat. Nos. 6,982,321 and 7,087,409.

The term “functional fragment” when used in reference to an antibody refers to a portion of the antibody including heavy or light chain polypeptides that retains some or all of the binding activity as the antibody from which the fragment was derived. Such functional fragments can include, for example, an Fd, Fv, Fab, F(ab′), F(ab)₂, F(ab′)₂, single chain Fv (ScFv), diabody, triabody, tetrabody and minibody. Other functional fragments can include, for example, heavy or light chain polypeptides, variable region polypeptides or CDR polypeptides or portions thereof so long as such functional fragments retain binding activity. Such antibody binding fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Pluckthun and Skerra, Meth. Enzymol., 178:497-515 (1989) and in Day, E. D., Advanced Immunochemistry, Second Ed., Wiley-Liss, Inc., New York, NY (1990). Antibody Engineering, Second Edition, Oxford University Press, 1995.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (λ) based on the amino acid sequence of the constant domains. Light chain amino acid sequences are well known in the art. A light chain can be a human light chain.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains can differ extensively in sequence between different antibodies. The variability in sequence is concentrated in the CDRs while the less variable portions in the variable domain are referred to as framework regions (FR). The CDRs of the light and heavy chains are primarily responsible for the interaction of the antibody with antigen. Numbering of amino acid positions used herein is according to the EU Index, as in Kabat et al. (1991) Sequences of proteins of immunological interest. (U.S. Department of Health and Human Services, Washington, D.C.) 5^{th} ed. A variable region can be a human variable region.

A CDR refers to one of three hypervariable regions (HI, H2 or H3) within the non-framework region of the immunoglobulin (Ig or antibody) VH β-sheet framework, or one of three hypervariable regions (LI, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., Biol. Chem. 252:6609-6616 (1977); Kabat, Adv. Prot. Chem. 32:1-75 (1978)). CDR region sequences also have been defined structurally by Chothia as those residues that are not part of the conserved β-sheet framework, and thus are able to adapt different conformations (Chothia and Lesk, Mol. Biol. 196:901-917 (1987)). Both terminologies are well recognized in the art. The positions of CDRs within a canonical antibody variable domain have been determined by comparison of numerous structures (Al-Lazikani et al., Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly skilled in the art.

### EXAMPLES

It will be appreciated that the following examples are intended to illustrate but not to limit the present disclosure. Various other examples and modifications of the foregoing description and examples will be apparent to a person skilled in the art after reading the disclosure without departing from the spirit and scope of the disclosure, and it is intended that all such examples or modifications be included within the scope of the appended claims. All publications and patents referenced herein are hereby incorporated by reference in their entirety.

### Example 1: Synthesis and In Vitro Activity of Compound of Formula (Ia)

The compounds of Formula (I), (Ia), and (II), can be synthesized by the methods provided in PCT/CN2020/07791, which is herein incorporated by reference in its entirety. In some embodiments, the syntheses of compounds described herein are accomplished using means described in the chemical literature, using the methods described herein, or by a combination thereof.

Cell lines and maintenance: Both cell lines (HCC827-ER1 and NCI-H1975) used for in vitro and in vivo studies were supplied by Crown Biosciences cell bank. HCC827-ER1 cells were maintained in RPMI medium supplemented with 10% heat inactivated fetal bovine serum+42 μM Erlotinib at 37 °C in an atmosphere of 5% CO₂ in air. NCI-H1975 cells were maintained in RPMI medium supplemented with 10% heat inactivated fetal bovine serum+100 ug/mL Hygromyocin at 37 °C in an atmosphere of 5% CO2 in air. The tumor cells were routinely subcultured twice weekly. The cells growing in an exponential growth phase were used for the in vitro cell potency and in vivo efficacy studies.

Cell Viability Assay: For cell cytotoxicity assays, cells were plated in 96-well plates and plating densities were determined on the basis of Crown Biosciences Cell line Database. Cells were plated at 2000 cells/well. Twenty four hours after plating, cells were treated with inhibitors at the indicated compound concentrations. On Day 5, cells were lysed with CellTiter-Glo® Luminescent Cell Viability Assay reagent (Promega) and luminescence was read using the En Vision Multi-plate reader. To calculate IC50s, a dose-response curve is generated using a nonlinear regression model with a sigmoidal dose response. IC50s were automatically generated by GraphPad Prism 8.0. The formula of surviving rate is shown below:

The surviving rate (%)=((LumTest article−LumMedium control)/(LumNone treated−LumMedium control))×100

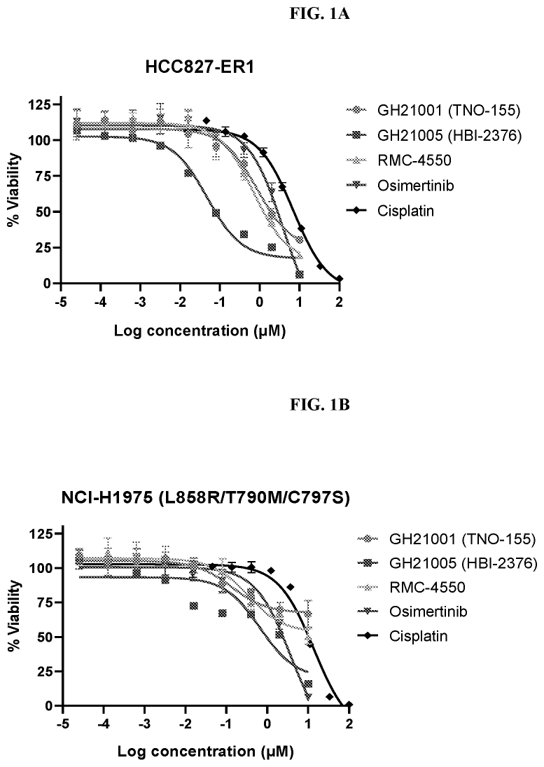

The in vitro test results are depicted in the log-linear graphs of FIG. 1A and FIG. 1B, and are summarized in Table 1.

TABLE 1
HBI-2376 demonstrates greater potency in cell
proliferation assay compared to TNO-155 and RMC-4550
Absolute IC50 (μM)

| | | NCI-H1975 |
|---|---|---|
| | HCC827-ER1 | (L858R/T790M/C797S) |
| GH21001(TNO-155) | 1.89 | >10 |
| GH21005(HBI-2376) | 0.08 | 0.96 |
| RMC-4550 | 1.23 | >10 |
| Osimertinib | 2.8 | 2.43 |
| Cisplatin | 7.17 | 10.33 |

### Example 2: Animal Models and In Vivo Treatments

Procedures involving the care and use of animals in this study were reviewed and approved by the Institutional Animal Care and Use Committee (IACUC) of Crown Biosciences prior to execution. During the study, the care and use of animals were conducted in accordance with the regulations of the Association for Assessment and Accreditation of Laboratory Animal Care (AAALAC). Animals (6-8 weeks) were obtained from Shanghai Lingchang Biotechnology Co., Ltd (Shanghai, China) and were allowed to acclimate prior to tumor cell inoculation.

GH21005 (the compound of Formula (Ia)) was supplied by Huyabio International, LLC. RMC-4550 was purchased from MCE, China. Osimertinib was purchased from Selleck, China.

All cell lines were maintained in culture and cells in exponential growth phase were harvested and quantitated by cell counter before tumor inoculation. HCC827-ER1 tumor cells (5×10⁷) in 0.1 ml of PBS mixed with Matrigel (1:1) inoculated into female Balb/C Nude animals, mean tumor approximately 325 mm³ at the start of treatment.

The date of randomization and treatment initiation was denoted as day 0. Tumor volumes were measured twice per week in two dimensions using a caliper, and the volume will be expressed in mm³ using the formula: V=(L×W×W)/2, where Vis tumor volume, Lis tumor length (the longest tumor dimension) and Wis tumor width (the longest tumor dimension perpendicular to L). Dosing volume was 10 mL/kg/day. GH21005 were dissolved in HP-β-CD was dissolved with 200 ml of 50 mM sodium citrate (pH=4.2). Anti-PD-1 and Rat IgG2a were diluted in PBS. RMC-4550 was diluted in 1% Captisol® in 50 mM sodium citrate (pH=5.0) 0.5% dosing solution of osimertinib was diluted in 2% DMSO/30% PEG300.

Upon termination, tumors were collected 2 hours post last dose. A portion of the tumor was minced and snap frozen immediately for protein isolation. Another portion of the tumor was fixed in 10% neutral buffered formalin prior to processing into paraffin blocks.

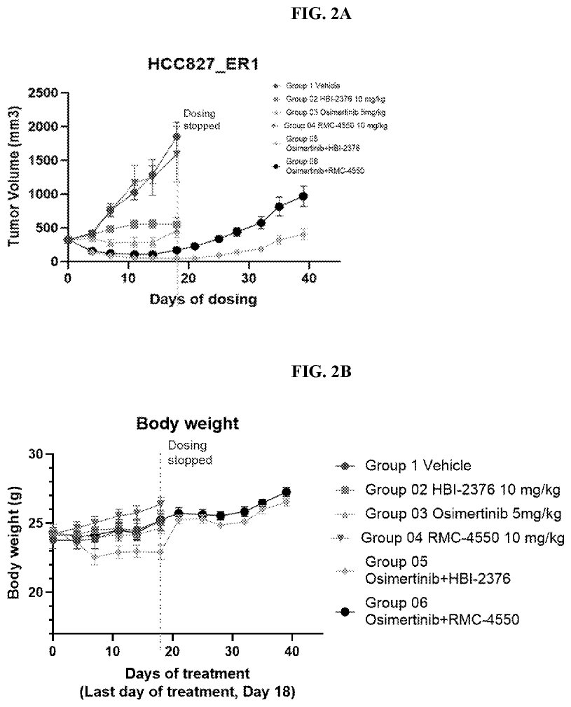

The results of these experiments are depicted in FIG. 2A and FIG. 2B (HCC827-ER1). The results show that treatment with only the compound of Formula (Ia) did not result in a reduction of tumor volume (see red line in FIG. 2A). Further, treatment with only osimertinib resulted in only a slight and temporary decrease in tumor volume, followed by growth in tumor volume prior to the termination of the treatment regime (see green line in FIG. 2A). However, treatment with a combination of the compound of Formula (Ia) and osimertinib resulted in significant and sustained reduction in tumor volume (see orange line in FIG. 2A). This reduction in tumor volume observed in the treatment group that received the combination of the compound of Formula (Ia) and osimertinib was itself lost following cessation of the treatment program. Collectively, these results evidence a synergistic effect between the compound of Formula (Ia) and osimertinib. FIG. 2A also showed the same synergistic relationship between RMC-4550 and osimertinib, evidencing that compounds of Formula (I) and Formula (II) exhibit a synergistic effect with osimertinib when administered to tumors.

### Example 3: Immunohistochemistry

Freshly collected tumor tissues were placed in 10% NBF and fixed for 24 hours at RT. Tumor tissue was trimmed and rinsed in running water. The specimens were transferred to the Vacuum Tissue Processor (Leica HistoCore PEARL) for dehydration, then embedded into FFPE blocks using Tissue embedding center (Leica HistoCore Arcadia Hot and Cold). FFPE blocks were sectioned with a manual rotary microtome (HistoCore MULTICUT, Leica), 4 μm thickness/section. Reagents for IHC and antigen retrieval including Bond™ Epitope Retrieval Solution 1 (Bond ER1), Bond™ Epitope Retrieval Solution 2 (Bond ER2), Bond™ Dewax Solution, Bond™ Wash. Primary antibodies Phospho-p44/42 MAPK (Erk1/2) (Thr202/Tyr204), CST 4370 and 44/42 MAPK (Erk1/2) (137F5) CST 4695 were used at 1:400 and 1:1000 dilution respectively. Anti-rabbit Poly-HRP-IgG (<25 μg/mL) containing 10% (v/v) animal serum in tris-buffered saline/0.09% ProClin™ 950 (ready-to-use) Leica DS9800 was used for secondary detection.

All stained sections were scanned with NanoZoomer-HT 2.0 Image system for 40× magnification High resolution picture for whole section was generated and further analyzed. All the images were analyzed with HALO™ platform.

IHC Scoring Method: The whole slide image was analyzed and necrosis and stroma areas were excluded. The intensity of specific staining were scored at four levels, 0 (negative), 1+ (weak staining), 2+ (medium staining), 3+ (strong staining). The percentages of tumor cells at different intensity levels were evaluated with H-score.

H-Score=(% at 0)×0+(% at 1)×1+(% at 2)×2+(% at 3)×3 (H-Score range is 0 to 300).

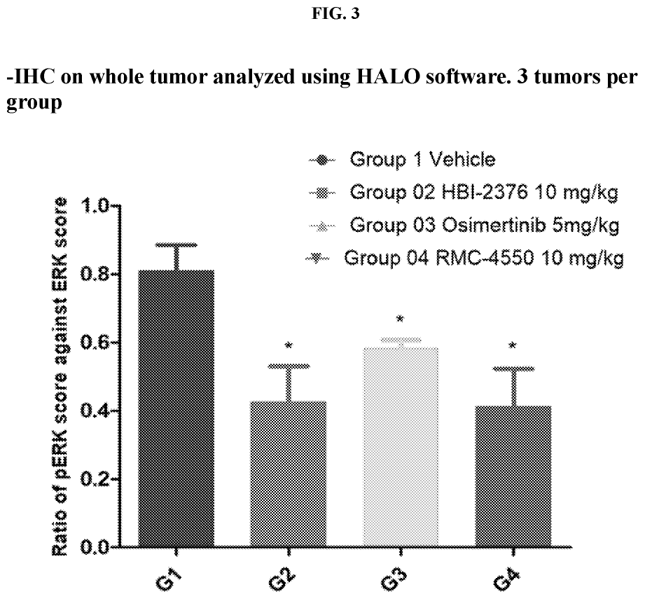

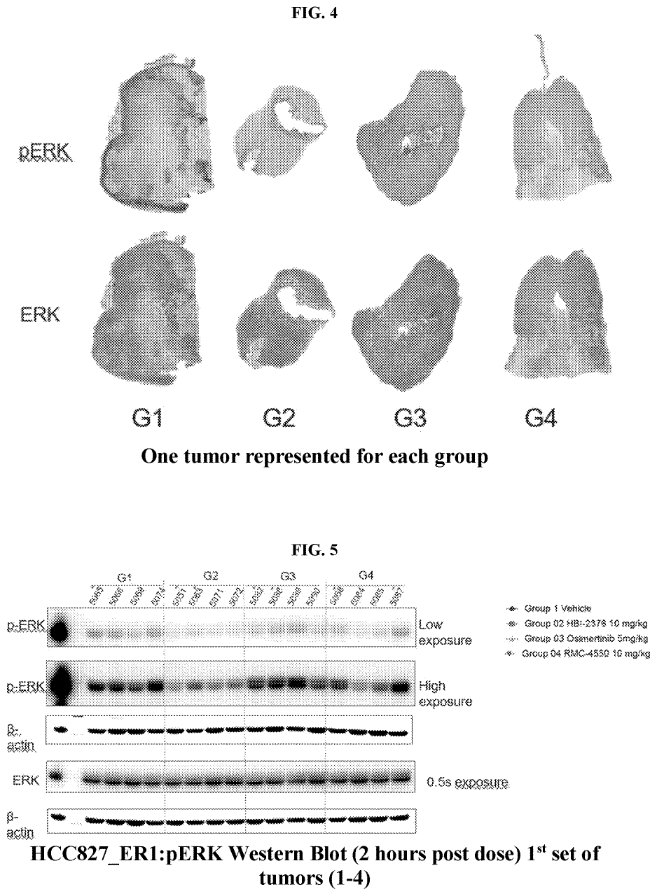

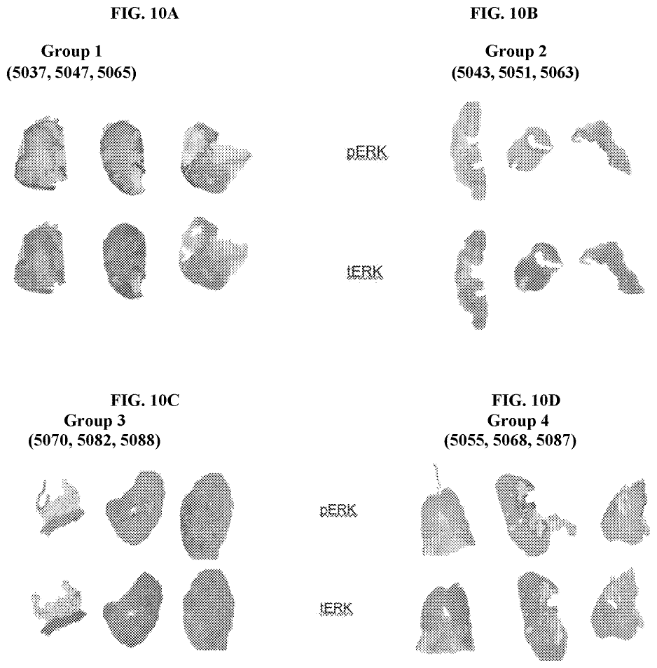

The ratio of pERK to ERK is represented in the graph in FIG. 3. Also see FIG. 4 and FIG. 10A, FIG. 10B, FIG. 10C and FIG. 10D for representative images.

### Example 4: Immunoblot Analysis

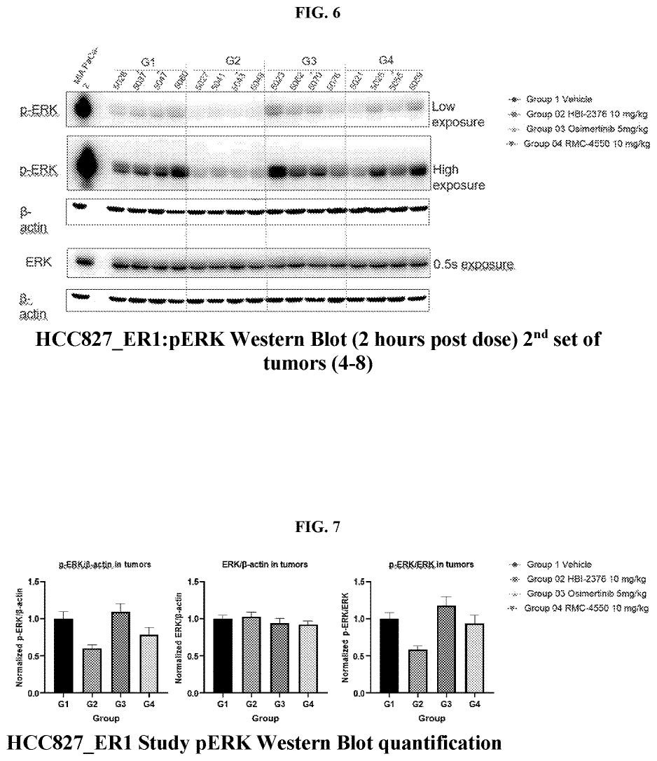

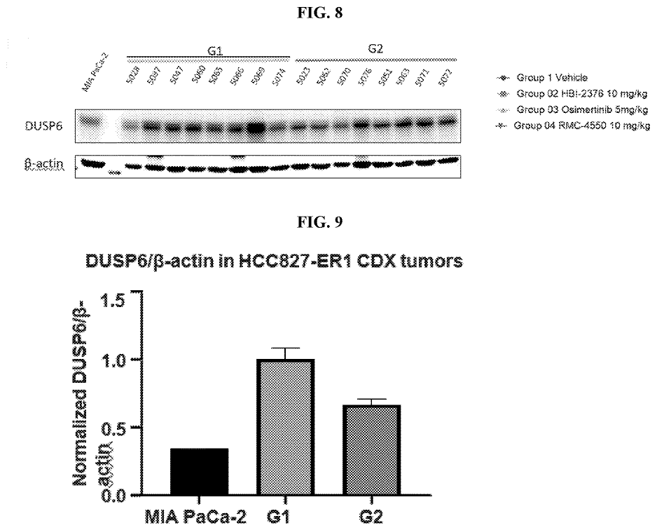

Tumors were harvested at the respective time point post dose and were snap frozen in liquid nitrogen. Tissue was ground in liquid nitrogen using a mortar and pestle and weighed. The volume of RIPA buffer containing phosphatase and protease inhibitors was added at 3 times the weight, samples were inverted and placed on ice for 30 min. Cell lysate was obtained by centrifugation at 14,000 g for 15 min at 4 °C and the supernatant was transferred to a fresh tube. Protein was quantified using Pierce BCA Protein Assay kit. 50 μg of protein was loaded in each well. Gels were transferred to preactivated PVDF and primary and secondary antibodies were diluted in TBST with 5% dry milk. Target proteins were detected with Tanon 5200 chemiluminescence image analysis system using ECL method. The ratio of pERK/ERK is measured and relative expression is quantified. See FIGS. 5, 6 and 7. The DUSP6/β-actin expression level is represented in FIGS. 8 and 9. Primary antibodies: DUSP6, Abcam ab76310, β-actin CST 3700S, p-ERK CST 4370s, ERK CST 4695s.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A combination comprising a compound of Formula (Ia), or a pharmaceutically acceptable salt or solvate thereof: 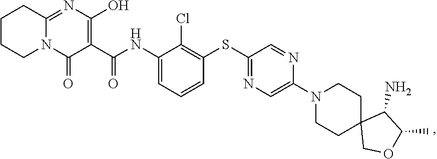 [SMILES: C[C@@H]1OCC2(CCN(c3cnc(Sc4cccc(NC(=O)c5c(O)nc6n(c5=O)CCCC6)c4Cl)cn3)CC2)[C@@H]1N]
and
an EGFR TK inhibitor (epidermal growth factor receptor tyrosine kinase inhibitor).

2. A method of treating cancer in a patient in need thereof, said method comprising administering to the patient a combination comprising a therapeutically effective amount of a compound of Formula (Ia), or a pharmaceutically acceptable salt or solvate thereof: 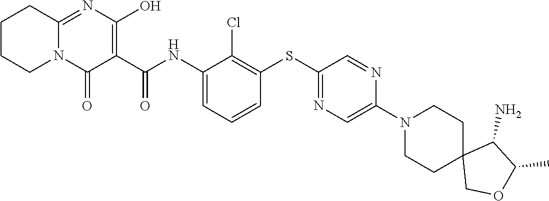 [SMILES: C[C@@H]1OCC2(CCN(c3cnc(Sc4cccc(NC(=O)c5c(O)nc6n(c5=O)CCCC6)c4Cl)cn3)CC2)[C@@H]1N]
and an EGFR TK inhibitor.

3. The method of claim 2, wherein said compound of Formula (Ia), or a pharmaceutically acceptable salt or solvate thereof, is administered to said patient in need from about 5 mg/kg to about 25 mg/kg.

4. The method of claim 2, wherein said compound of Formula (Ia), or a pharmaceutically acceptable salt or solvate thereof is administered to said patient in need at about 5 mg/kg, about 10 mg/kg, about 15 mg/kg, about 20 mg/kg, or about 25 mg/kg.

5. The method of claim 2, wherein said EGFR TK inhibitor is a small molecule compound, a nucleic acid, a peptide, a protein, an antibody, a peptibody, a diabody, a single-chain variable fragment (ScFv), or a minibody.

6. The method of claim 2, wherein said EGFR TK inhibitor is selected from erlotinib, afatinib, gefitinib, osimertinib, dacomitinib, icotinib, rociletinib, olmatinib, tarloxotinib, TAK-788, amivantamab (JNJ-6372), or AC0010.

7. The method of claim 6, wherein said EGFR TK inhibitor is osimertinib.

8. The method of claim 2, wherein said cancer is squamous cell carcinoma, nonsquamous cell carcinoma, non-small cell lung cancer (NSCLC), small cell lung cancer, melanoma, hepatocellular carcinoma, renal cell carcinoma, ovarian cancer, head and neck cancer, urothelial cancer, breast cancer, prostate cancer, glioblastoma, colorectal cancer, pancreatic cancer, lymphoma, leiomyosarcoma, liposarcoma, synovial sarcoma, or malignant peripheral sheath tumor (MPNST).

9. The method of claim 8, wherein said method comprises administering said compound of Formula (Ia), or a pharmaceutically acceptable salt or solvate thereof; and said EGFR TK inhibitor to said patient following treatment with at least one anti-cancer therapy, wherein said anti-cancer therapy is chemotherapy, radiotherapy, surgery, targeted therapy, immunotherapy, or a combination thereof.

10. A combination comprising:
(i) a therapeutically effective amount of a SHP2 inhibitor of Formula (I), or a pharmaceutically acceptable salt or solvate thereof: 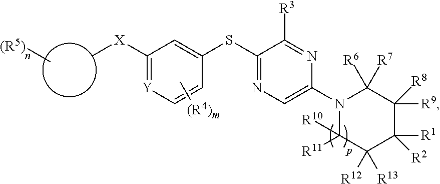
wherein,
R¹ and R² are each independently selected from H, D, halogen, —CN, —C(O)OH, —CHO, —OH, —NO₂, and substituted or unsubstituted —NH₂, C₁-C₁₀ alkyl, C₁-C₁₀ alkylamino, C₁-C₁₀ alkoxy, C₃-C₁₂ cycloalkyl, C₃-C₁₂ cycloalkyloxy, 3-12 membered heterocyclic group, or C₆-C₁₀ aryl group, 5-10 membered heteroaryl group; or R¹ and R² form a 3-8 membered saturated or unsaturated cycloalkyl or heterocyclic group, wherein optionally, the 3-8 membered saturated or unsaturated cycloalkyl or heterocyclic group has one to three-OH, —NH₂, —CN, NO₂, halogen, C₁-C₁₀ alkyl, C₁-C₁₀ alkoxy, C₁-C₁₀ alkylamino, C₃-C₁₂ cycloalkyl, C₆-C₁₀ aryl or 5-10 membered heteroaryl;
R³ is selected from H, D, or —NH₂;
X is selected from a bond, —NH—, or —C(O)NH—;
Y is selected from N or CR¹³, wherein R¹³ is selected from H, D, —OH, —CN, halogen, C₁-C₁₀ alkyl group, C₁-C₁₀ alkoxy, C₃-C₁₂ cycloalkane amino, C₁-C₁₀ alkylamino, C₃-C₁₂ cycloalkyl, 3-8 membered heterocyclic group, halogenated C₁-C₁₀ alkylamino, or C₆-C₁₀ aryl or 5-10 membered heteroaryl group, the heterocyclic group or heteroaryl group optionally contains one to four heteroatoms, and the heteroatoms are selected from S, O, N or NH;
each R⁴ is independently selected from H, D, halogen, —CN, —C(O)OH, —CHO, —OH, —NO₂, —C(O)NHR¹⁴ or —NHC(O)R¹⁵, wherein R¹⁴ and R¹⁵ are selected from substituted or unsubstituted C₁-C₁₀ alkylamino, C₃-C₁₂ cycloalkyl, C₆-C₁₀ aryl or 5-10 membered heteroaryl, wherein each of R¹⁴ and R¹⁵ is optionally substituted with a substituent selected from C₁-C₁₀ alkyl, a halogen, —NH₂, —CN, —C(O)OH, —CHO, —OH, —NO₂, C₁-C₁₀ alkoxy, C₁-C₁₀ alkylamino, C₃-C₁₂ cycloalkyl; 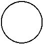
is selected from C₆-C₁₀ aryl, 5-10 membered heteroaryl, C₄-C₁₂ cycloalkyl, 3-12 membered heterocyclic group, C₆-C₁₄ bridged ring group or spiro ring group, or C₆-C₁₄ bridged heterocyclic group or spiro heterocyclic group; wherein the 5-10 membered heteroaryl, 3-12 membered heterocyclic group, C₆-C₁₄ bridged heterocyclic group or spiro heterocyclic group contains one to three heteroatoms or groups selected from N, NH, O, S, C(O), or S(O);
each R⁵ is independently selected from H, D, halogen, —CN, —C(O)OH, —CHO, —OH, —NO₂, aminoacyl, substituted or unsubstituted following groups: C₁-C₁₀ alkyl, C₁-C₁₀ alkylamino, C₁-C₁₀ alkoxy, —NH₂, C₃-C₁₂ cycloalkyl, 3-12 membered heterocyclic group, C₆-C₁₀ aryl or 5-10 membered heteroaryl, the substitution is selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaromatic group, C₆-C₁₀ aryl group or 3-12 membered heterocyclic group substituted by one or more substituents; or any two adjacent R⁵ form a 3-6 membered saturated or unsaturated ring, and optionally the 3-6 membered saturated or unsaturated ring is substituted with one to three-OH, —NH₂, —CN, halogen, C₁-C₁₀ alkyl, C₁-C₁₀ alkoxy, C₃-C₁₂ cycloalkylamino, C₁-C₁₀ alkylamino, C₃-C₁₂ cycloalkyl, halogenated C₁-C₁₀ alkylamino, C₆-C₁₀ aryl or 5-10 member heteroaryl;
R⁶, R⁷, R⁸, R⁹, R¹⁰, R¹¹, R¹², and R¹³ are each independently selected from H, D, halogen, —CN, —C(O)OH, —CHO, —OH, —NO₂, substituted or unsubstituted the group selected from —NH₂, C₁-C₁₀ alkyl, C₁-C₁₀ alkylamino, C₁-C₁₀ alkoxy, C₃-C₁₂ cycloalkyl, C₃-C₁₂ cycloalkyloxy group, 3-12 membered heterocyclic group, C₆-C₁₀ aryl, and 5-10 membered heteroaryl, the substitution is selected from C₁-C₁₀ alkyl, C₃-C₁₂ cycloalkyl, 3-12 membered heterocyclic group, halogen, —NH₂, —CN, —C(O)OH, —CHO, —OH, —NO₂, hydroxy-C₁-C₁₀ alkyl, C₁-C₁₀ alkoxy, C₁-C₁₀ alkylamino, 5-10 membered heteroaryl or C₆-C₁₀ aryl;
m is 0, 1, 2 or 3;
n is 0, 1, 2 or 3; and
p is 0, 1, or 2; and
(ii) a therapeutically effective amount of an EGFR TK inhibitor.

11. The combination of claim 10, wherein said combination comprises from about 5 mg to about 100 mg of said SHP2 inhibitor of Formula (I), or a pharmaceutically acceptable salt or solvate thereof.

12. The combination of claim 10, wherein said combination comprises about 5 mg, about 10 mg, about 20 mg, about 30 mg, about 40 mg, or about 50 mg of said SHP2 inhibitor of Formula (I), or a pharmaceutically acceptable salt or solvate thereof.

13. The combination of claim 10, wherein said EGFR TK inhibitor is a small molecule compound, a nucleic acid, a peptide, a protein, an antibody, a peptibody, a diabody, a minibody, a single chain variable fragment (ScFv), or a variant thereof.

14. The combination of claim 10, wherein said EGFR TK inhibitor is selected from erlotinib, afatinib, gefitinib, osimertinib, dacomitinib, icotinib, rociletinib, olmatinib, tarloxotinib, TAK-788, amivantamab (JNJ-6372), or AC0010.

15. The combination of claim 10, wherein said EGFR TK inhibitor is osimertinib.
